(19) United States
(12) Patent Application Publication
(10) Pub. No.: US 2026/0290625 A1
(43) Pub. Date: Sep. 24, 2026

(54) SYSTEMS AND METHODS FOR TRIAGE-BASED ROUTING TO GENERATIVE MODELS WITH LOG-SPACE PROBABILITY-DISTRIBUTION AGGREGATION AND RANK-FREQUENCY BOOSTING

(71) Applicant: ODIGGO, INC., Santa Clara, CA (US)

(72) Inventors: Henry DUONG, Santa Clara, CA (US); Chaitanya GHARPURE, Santa Clara, CA (US)

(73) Assignee: ODIGGO, INC., Santa Clara, CA (US)

(21) Appl. No.: 19/697,038

(22) Filed: Jun. 3, 2026

Related U.S. Application Data
(63) Continuation of application No. 19/368,376, filed on Oct. 24, 2025.
Application No. 19/368,376 is a continuation of application No. 19/253,887, filed on Jun. 29, 2025, now Pat. No. 12,525,363.
Application No. 19/253,887 is a continuation-in-part of application No. 18/902,532, filed on Sep. 30, 2024, now Pat. No. 12,347,573.
Application No. 18/902,532 is a continuation-in-part of application No. 18/823,175, filed on Sep. 3, 2024, now Pat. No. 12,254,966.
(60) Provisional application No. 63/817,108, filed on Jun. 3, 2025.

(51) Int. Cl. G16H 80/00; G16H 50/20

(52) U.S. Cl. CPC G16H 80/00; G16H 50/20

(57) ABSTRACT

A system routes a query to a plurality of generative language models and aggregates their outputs to produce a final answer. A triage model determines a task type and a plurality of specialties from the query and maps individual specialties to corresponding generative language models, at least one of which is a locally hosted model that processes the query within an infrastructure that retains the query. Individual generative language models return a probability distribution over a set of candidate answers. A combined probability distribution is computed as a weighted sum, in logarithmic space, of the probability distributions under expert weights that are positive and sum to one, and is normalized. A final probability distribution is determined from the normalized probability distribution. A consensus model emits the final answer based on the final probability distribution and on a rationale received from individual generative language models.

This application is a continuation-in-part of U.S. application Ser. No. 19/368,376, filed Oct. 24, 2025, and claims the benefit under 35 U.S.C. § 119(e) of U.S. Provisional Application No. 63/817,108, filed Jun. 3, 2025. The disclosures of all of the above-referenced applications are incorporated herein by reference in their entireties.

### TECHNICAL FIELD

The technology disclosed relates to artificial intelligence type computers and digital data processing systems and corresponding data processing methods and products for emulation of intelligence (i.e., knowledge based systems, reasoning systems, and knowledge acquisition systems); and including systems for reasoning with uncertainty (e.g., fuzzy logic systems), adaptive systems, machine learning systems, and artificial neural networks. Specifically, the present disclosure relates to machine learning systems, and more particularly to systems and methods that generate a final answer to a query by aggregating probability distributions produced by an ensemble of generative language models.

### BACKGROUND

Generative language models are increasingly used to assist with complex reasoning tasks, including tasks in regulated fields such as medicine, where a system receives a query and is expected to return an answer drawn from a set of possible answers. A clinical query, for example, may describe a patient and ask for a most appropriate next step, and the desired output is not merely a single answer but an answer accompanied by some indication of how confident the system is in that answer. Many deployments address this expectation by relying on a single generative language model that is asked to process the query and report an answer together with a confidence in that answer. Such single-model deployments are attractive because they are simple to operate, yet they couple the entire system to the behavior of one model. The field has therefore explored techniques that seek deeper reasoning from a single model, such as prompting the model to reason step by step before answering, and techniques that route a query among internal sub-experts of a single model, such as a mixture-of-experts arrangement. These techniques operate within the boundary of one model, and the outputs they produce continue to reflect the strengths, the weaknesses, and the particular calibration of that one model.

Reliance on a single model, however configured, creates several difficulties in fields that demand sustained reliability. Generative language models are revised, replaced, or made available on changing terms with considerable frequency, so a workflow built around any one model is exposed to obsolescence and to shifts in access, cost, and behavior over time. A single model also tends to express a confidence that does not consistently match its observed accuracy, and an answer that is reported with high confidence yet is incorrect can mislead a decision maker in a high-stakes setting. Many capable models are, in addition, available only through external services, which requires a query to be transmitted away from the local infrastructure of the operator and raises concerns in settings where the query contains sensitive information. There is, accordingly, a need for an approach to decision support that does not bind the operator to the fortunes of any single model, that yields an answer whose reported confidence more faithfully reflects its accuracy, and that can be operated within local infrastructure when the sensitivity of a query so requires.

### SUMMARY

The present disclosure relates to systems and methods for generating a final answer to a query using an ensemble of generative language models. In some implementations, a triage model determines, based on a query, a task type and a plurality of specialties and maps each specialty to a corresponding expert model of a plurality of expert models, at least one of which is a locally hosted model that processes the query within an infrastructure that retains the query. Each expert model returns a probability distribution over a set of candidate answers. The system computes a combined probability distribution as a weighted sum, in logarithmic space, of the probability distributions under expert weights that are positive and sum to one, normalizes the combined probability distribution, and determines a final probability distribution based at least in part on the normalized probability distribution. A consensus model that is separate from the expert models emits a final answer to the query based at least in part on the final probability distribution and on a rationale received from each expert model, and the system provides the final answer and a representation of the final probability distribution to a computing device as a decision support output. Corresponding methods and non-transitory computer-readable storage media are also described.

### BRIEF DESCRIPTION OF THE DRAWINGS

A more complete appreciation of this disclosure and many of the attendant advantages thereof will be readily obtained as the same becomes better understood by reference to the following detailed description when considered in connection with the accompanying drawings, wherein:

FIG. 1 is a block diagram of an operating environment of a consensus-based decision support system, according to some implementations.

FIG. 2 is a block diagram of the consensus-based decision support system, illustrating a triage model, a plurality of expert models, a probability-weighting module, a cascade-boosting module, and a consensus model, according to some implementations.

FIG. 3 is a block diagram illustrating the probability-weighting module that computes a combined probability distribution as a weighted sum in logarithmic space and normalizes the combined probability distribution, according to some implementations.

FIG. 4 is a block diagram illustrating the cascade-boosting module that constructs a rank-frequency table, applies cascade weights, applies a boost scaler, and produces a final probability distribution, according to some implementations.

FIG. 5 is a block diagram illustrating the consensus model that emits a final answer based on the final probability distribution, a respective rationale, and a respective specialty, according to some implementations.

FIG. 6 is a flow diagram of a process to produce a final answer to a query using the consensus-based decision support system, according to some implementations.

FIG. 7 is a flow diagram of a process to determine the final probability distribution using cascade boosting, according to some implementations.

FIG. 8 is a block diagram illustrating selection and substitution of expert models from a model pool, according to some implementations.

FIG. 9 is a block diagram of a server hardware architecture for implementing the consensus-based decision support system, according to some implementations.

FIG. 10 is a schematic representation of an encoder-decoder architecture.

FIG. 11 shows an overview of an attention mechanism added onto an RNN encoder-decoder architecture.

FIG. 12 is a schematic representation of the calculation of self-attention showing one attention head.

FIG. 13 is a depiction of several attention heads in a Transformer block.

FIG. 14 is an illustration that shows how one can use multiple workers to compute the multi-head attention in parallel, as the respective heads compute their outputs independently of one another.

FIG. 15 is a portrayal of one encoder layer of a Transformer network.

FIG. 16 shows a schematic overview of a Transformer model.

FIG. 17 is a depiction of a Vision Transformer (ViT).

FIG. 18 illustrates a processing flow of the Vision Transformer (ViT).

FIG. 19 shows example software code that implements a Transformer block.

### DETAILED DESCRIPTION

In the drawings, like reference numerals designate identical or corresponding parts throughout the several views. Further, as used herein, the words “a,” “an” and the like generally carry a meaning of “one or more,” unless stated otherwise.

Furthermore, the terms “approximately,” “approximate,” “about,” and similar terms generally refer to ranges that include the identified value within a margin of 20%, 10%, or preferably 5%, and any values therebetween.

### Indicia of Novelty, Inventiveness, Non-Obviousness, and Subject-Matter Eligibility

Organizations increasingly use a generative language model to answer questions that once required a panel of human specialists, such as which treatment fits a patient or which action resolves an incident. A single model now answers such a question well, so the ability of any one model is no longer the limiting factor. The reliability of depending on that one model is. When an organization builds its workflow on one model, the workflow inherits every weakness of that model. The model is revised, replaced, or repriced by its provider, and the workflow that depended on it breaks. The model reports high confidence in an answer that is wrong, and the person relying on it acts on that wrong answer. The model is available only through an external service, so a question that contains confidential information has to be sent outside the organization to be answered. Each of these failures comes from depending on a single model, so choosing a better single model does not remove any of them.

The technology disclosed removes these failures by answering each question with several models instead of one, and by combining their answers in a way that a single model cannot replicate. A triage component reads the incoming question, identifies what kind of question it is, and selects a set of expert models suited to it. Each expert model does not return one answer. It returns a full set of probabilities spread across the same list of possible answers, so that the relative support each possible answer receives is preserved rather than collapsed into a single guess. A probability-weighting component then merges these sets of probabilities by combining them in logarithmic form under per-model weights that are positive and add up to one. Combining in logarithmic form takes a weighted geometric blend of the sets, which strengthens an answer that the models agree on and softens the influence of a model whose answer diverges from the rest, so that genuine disagreement produces a more even spread rather than false certainty.

The technology then captures a signal that simple merging leaves behind. A correct answer often appears second or third across several models without being the top choice of any one of them, and a method that keeps only top choices discards it. A boosting component records how often each possible answer lands at each rank across the models, gives a higher rank more influence than a lower rank by a weight that halves at each step down, scales that contribution by an adjustable amount, adds it to the merged probability of each answer, and converts the result back into a final set of probabilities. An answer that several models quietly support rises to where a decision maker can see it. A separate consensus component then reads this final set of probabilities together with the written reasoning each expert model produced, and selects the final answer, and it can favor the answer carried by the strongest reasoning over the answer with the highest number. The final decision reflects the reasoning behind the probabilities, not the arithmetic alone.

### TECHNICAL EFFECTS

These steps deliver three benefits that an organization can rely on, and each benefit is a property of how the technology works rather than of any one model. First, the final set of probabilities reports a confidence that matches how often the answer is actually correct, more closely than any single model does, because the technology blends the independent outputs of several models instead of trusting one model's report of its own confidence. A reliable confidence is what prevents a confident wrong answer from being acted upon. Second, the set of models is reconfigurable. The technology replaces one expert model with a different one without changing the consensus component, and the list of possible answers and the form of the final result stay the same across the change, so the obsolescence of a model becomes a simple swap rather than a rebuild, and an operator can tune the set of models for cost, speed, or accuracy. Third, a confidential question can stay inside the organization, because at least one expert model runs on the organization's own infrastructure and answers the question without sending it to any outside service.

The technology achieves these benefits through a concrete and specific sequence of computational operations performed on machine-generated probability distributions. Earlier approaches operate within the boundary of a single model. Asking one model to reason step by step still relies on that one model. Routing a question among internal portions of one model still relies on that one model. Such approaches do not combine independent probability distributions generated by separate models. The technology disclosed instead carries out a defined pipeline in which each model expresses its judgment across a shared set of candidate answers, the probability distributions are blended in logarithmic form under weights that are positive and sum to one, the blended result is reweighted according to recurrence of candidate answers at high ranks, and a separate consensus component selects the final answer using the resulting probability distribution and associated rationale data. The system runs on one or more processors and storage media that store the triage component, the expert models, the probability-weighting component, the boosting component, and the consensus component, and the processors carry out the stored computational operations within infrastructure that retains the query. The disclosed arrangement improves computer-implemented decision-support operation by producing probability-associated output data from full probability distributions of independent models, by enabling substitution of an expert model without rebuilding the consensus component, and by permitting at least a portion of sensitive-query processing to remain within local infrastructure.

### System Environment

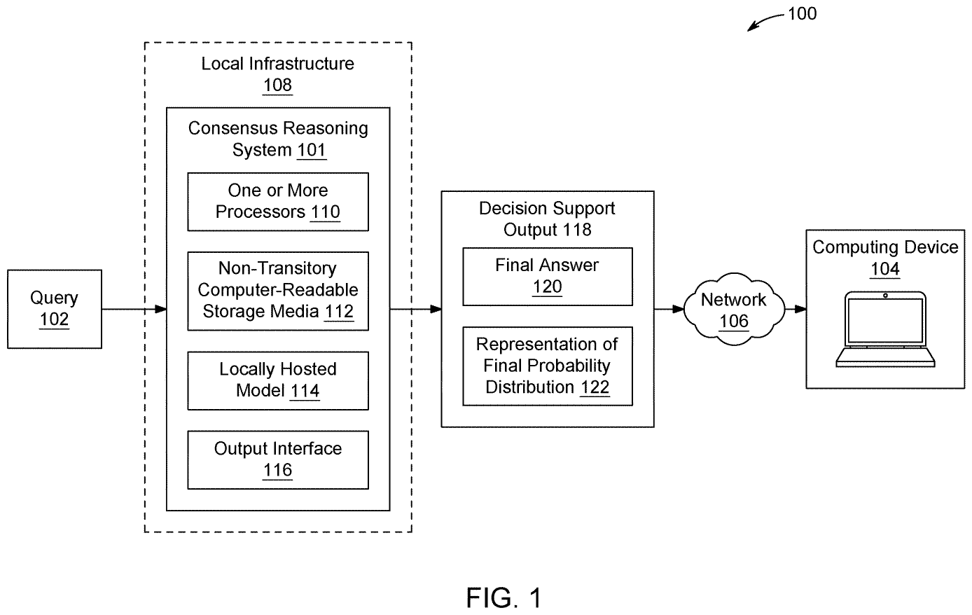

FIG. 1 illustrates an operating environment 100 of a consensus reasoning system 101. The consensus reasoning system 101, also referred to as the system 101, is configured to receive a query 102 and to produce, in response to the query 102, a decision support output 118 that is provided to a computing device 104. The system 101 may be implemented as software executing on a server, a dedicated appliance, a distributed service across multiple machines, or a similar computing arrangement configured to process the query 102 and generate the decision support output 118.

The query 102 is an input that identifies a problem to be resolved and is associated with a set of candidate answers from which an answer may be drawn. The query 102 is configured to be processed by the system 101 to yield a final answer 120 together with a representation of a final probability distribution 122. In some implementations, the query 102 comprises a medical query that describes a patient and requests a most appropriate course of action, and the set of candidate answers comprises a set of mutually exclusive treatment options or diagnoses. In other implementations, the query 102 comprises a technical support query, a financial analysis query, a legal research query, a classification query, or another query for which a set of candidate answers can be defined. The query 102 may be expressed in natural language, in structured form, or in a combination thereof, and may include accompanying context such as a description of a subject, a set of measurements, or a set of constraints relevant to the problem to be resolved.

The system 101 operates within a local infrastructure 108. The local infrastructure 108 comprises computing resources controlled by an operator of the system 101 and is configured to retain the query 102 such that at least a portion of processing of the query 102 occurs within the local infrastructure 108. The local infrastructure 108 preserves confidentiality of the query 102 where the query 102 contains sensitive information and reduces dependence on an external service whose availability, cost, or behavior may not be controlled by the operator. The local infrastructure 108 may comprise, for example, an on-premises server, a private data center, a private cloud allocation dedicated to the operator, an edge computing node, or a similar computing resource. In some implementations, the local infrastructure 108 hosts every model used by the system 101. In other implementations, the local infrastructure 108 hosts at least one model while one or more additional models are accessible through an external service, provided that the locally hosted model 114 processes the query 102 within the local infrastructure 108.

The system 101 comprises one or more processors 110. The one or more processors 110 are configured to execute instructions that cause the system 101 to perform operations attributed to the system 101, including processing the query 102, generating intermediate data objects, and producing the decision support output 118. The one or more processors 110 may comprise, for example, a central processing unit, a graphics processing unit, a digital signal processor, a tensor processing unit, a field-programmable gate array, an application-specific integrated circuit, a system-on-chip, or a combination thereof. In some implementations, the one or more processors 110 comprise a plurality of processors distributed across a plurality of machines within the local infrastructure 108.

The system 101 further comprises one or more non-transitory computer-readable storage media 112. The one or more non-transitory computer-readable storage media 112 store instructions executed by the one or more processors 110 and store data objects produced by the system 101 in the course of processing the query 102. The one or more non-transitory computer-readable storage media 112 may comprise, for example, random access memory, read-only memory, electrically erasable programmable read-only memory, flash memory, a solid-state drive, a hard disk drive, an optical storage medium, a magnetic storage device, or a combination thereof. In some implementations, the one or more non-transitory computer-readable storage media 112 are configured as a distributed storage system across a plurality of machines within the local infrastructure 108. As used herein, a model refers to a configured machine-learning model implemented as instructions stored by the one or more non-transitory computer-readable storage media 112 and executed by the one or more processors 110.

The system 101 further comprises the locally hosted model 114. The locally hosted model 114 is configured to process the query 102 within the local infrastructure 108. The locally hosted model 114 may comprise, for example, a generative language model deployed on the one or more processors 110, a model trained or fine-tuned for a particular area of analysis, a quantized model selected to reduce computational cost, or a similar model. The locally hosted model 114 allows the system 101 to process the query 102 without requiring transmission of the query 102 to an external service. In some implementations, the locally hosted model 114 is one of several locally hosted models. In other implementations, the locally hosted model 114 operates with one or more additional models that are not shown in FIG. 1.

The system 101 further comprises an output interface 116. The output interface 116 is configured to provide the decision support output 118 from the system 101 toward the computing device 104. The output interface 116 may comprise, for example, an application programming interface, a network interface, a message queue, a user interface component, or a similar interface. The output interface 116 may format the decision support output 118 according to a protocol expected by the computing device 104.

The system 101 communicates with the computing device 104 over one or more networks 106. The one or more networks 106 may comprise one or more wired networks, one or more wireless networks, or a combination thereof, and may employ Transmission Control Protocol/Internet Protocol, Hypertext Transfer Protocol, Wi-Fi, a cellular communication standard, or another communication protocol. The computing device 104 receives the decision support output 118 and presents the decision support output 118 to a user. The computing device 104 may comprise, for example, a laptop computer, a desktop computer, a tablet computer, a smartphone, a workstation, or a similar device capable of communicating over the one or more networks 106 and presenting the decision support output 118.

The decision support output 118 comprises the final answer 120 and the representation of the final probability distribution 122. The final answer 120 is the answer to the query 102 produced by the system 101. The representation of the final probability distribution 122 conveys, for individual candidate answers of the set of candidate answers, a probability that the system 101 associates with the corresponding candidate answer. The representation of the final probability distribution 122 may comprise, for example, two or more candidate answers ranked according to the final probability distribution, a probability value associated with individual candidate answers, a structured representation of probabilities assigned to candidate answers, or a similar representation. By providing the representation of the final probability distribution 122 together with the final answer 120, the system 101 provides the decision support output 118 in a manner that allows a user to consider both the final answer 120 and the confidence information associated with candidate answers.

### Consensus Reasoning System

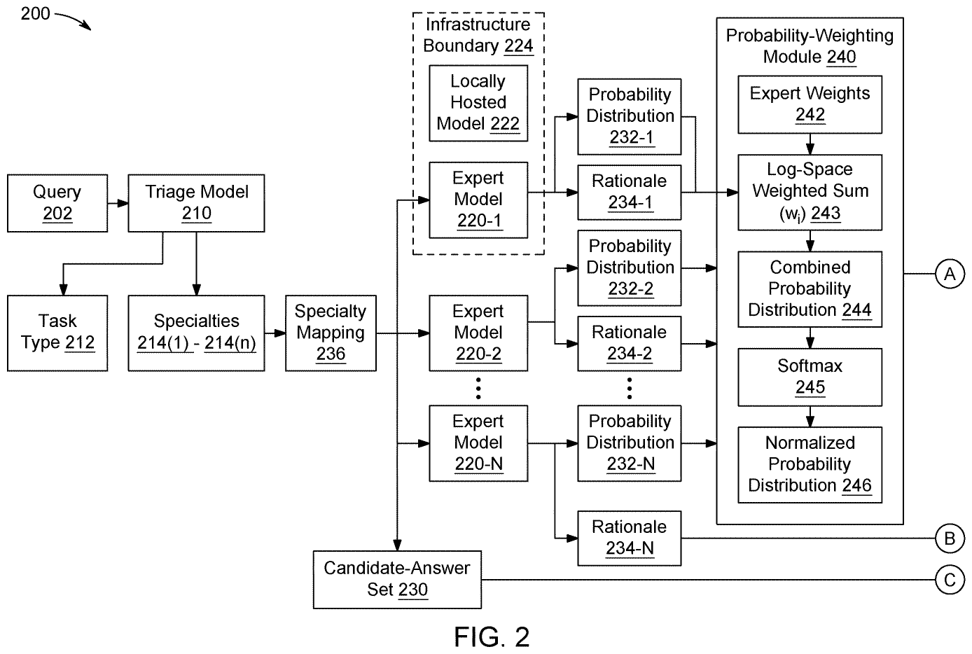

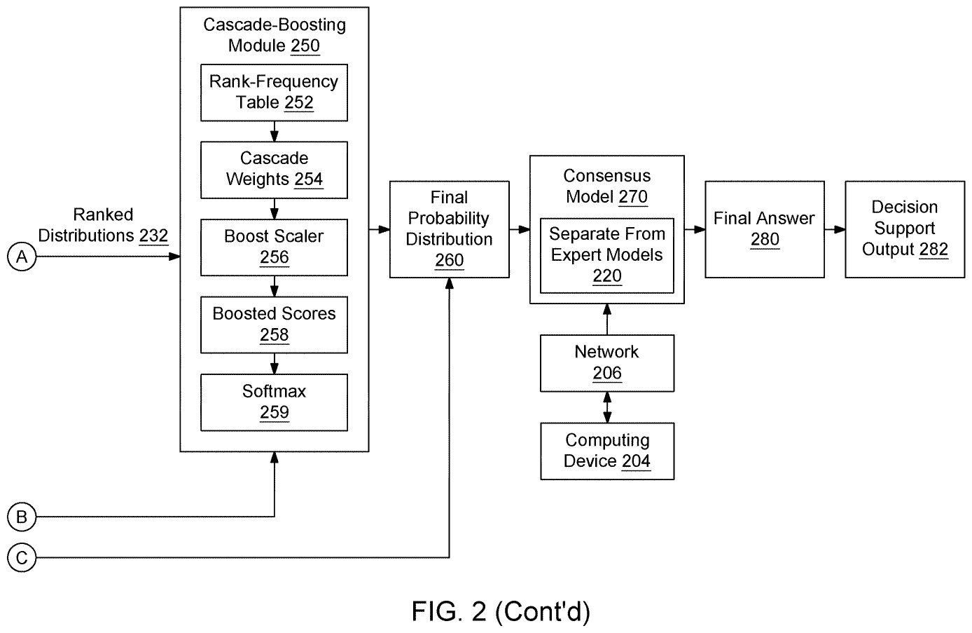

FIG. 2 illustrates the consensus reasoning system 200 that produces a final answer 280 in response to a query 202. The system 200 receives the query 202 and routes the query 202 to a plurality of expert models, obtains from each expert model a probability distribution over a shared set of candidate answers, aggregates the probability distributions into a final probability distribution 260, and emits the final answer 280 through a consensus model 270 that is separate from the expert models. The system 200 thereby generates the final answer 280 from coordinated outputs of a plurality of expert models rather than from an output of any single model.

The query 202 enters a triage model 210. The triage model 210 is a model configured to determine, based on the query 202, a task type 212 and a plurality of specialties 214(1) through 214 (n), and to map the specialties to corresponding expert models. The objective of the triage model 210 is to identify a task category associated with the query 202 and to select a set of expert models corresponding to specialties determined for the query 202. The task type 212 is a category of the query 202, such as a diagnosis task, a treatment task, a classification task, or an analysis task. Each specialty 214 is a specialty domain selected by the triage model 210 as relevant to the query 202, such as a medical specialty in a medical application or a technical domain in a technical application. The triage model 210 performs a specialty mapping 236 that associates individual specialties 214(1) through 214 (n) with corresponding expert models 220-1 through 220-N. In some implementations the triage model 210 extracts one or more contextual signals from the query 202 and selects the specialties 214 based on the one or more contextual signals, and in some implementations the triage model 210 adds an expert model to, or removes an expert model from, the set of expert models based on the task type 212.

The specialty mapping 236 populates a plurality of expert models 220-1, 220-2, through 220-N. Each expert model 220 is an independently configured generative language model assigned to a mapped specialty and configured to process the query 202 according to that mapped specialty. The objective of using a plurality of expert models 220, rather than a single model, is to process the query 202 using multiple specialty-specific model configurations and to reduce dependence of the system 200 on an output, probability calibration, or operational behavior of any single model. Each expert model 220 may comprise, for example, a publicly available generative language model, an open-source model, a model fine-tuned for a particular specialty, or a similar model, and the expert models 220 may be distinct models or distinct configurations of a common model. At least one expert model, shown as a locally hosted model 222, executes within an infrastructure boundary 224 and processes the query 202 within the infrastructure that the infrastructure boundary 224 delimits, so that the query 202 provided to the locally hosted model 222 is not transmitted to a service external to the infrastructure. The expert models 220 are independently substitutable, such that one expert model is replaced by a different model without reconfiguration of the consensus model 270.

Each expert model 220 processes the query 202 over a candidate-answer set 230. The candidate-answer set 230 is the shared set of candidate answers over which each expert model 220 defines its output, and in some implementations comprises a set of mutually exclusive answer options. Each expert model 220 returns a probability distribution and a rationale. The probability distributions 232-1, 232-2, through 232-N are the outputs by which each expert model 220 assigns, across the candidate-answer set 230, a probability to individual candidate answers, the probabilities assigned by an individual expert model summing to one. The objective of returning a probability distribution 232, rather than a single answer with a confidence score, is to preserve the relative probability support that the expert model 220 assigns to each candidate answer, which a single answer would discard. The rationales 234-1, 234-2, through 234-N are reasoning outputs that each expert model 220 returns in association with its probability distribution 232, and may comprise, for example, a clinical reasoning, a scientific reasoning, an elimination reasoning that identifies excluded candidate answers, or a similar reasoning output.

The probability distributions 232 enter a probability-weighting module 240. The probability-weighting module 240 is configured to combine the probability distributions 232 into a single combined probability distribution 244. The objective of the probability-weighting module 240 is to combine the independently generated probability distributions of the expert models 220 into one distribution that reflects aggregated probability support for each candidate answer. The probability-weighting module 240 applies expert weights 242, which are per-expert weights that are positive and sum to one and that govern the contribution of each expert model 220 to the combined probability distribution 244. In some implementations the expert weights 242 are adjustable to vary the contribution of a corresponding expert model 220. The probability-weighting module 240 performs a log-space weighted sum 243, which combines the probability distributions 232 as a weighted sum in logarithmic space under the expert weights 242, thereby computing a weighted geometric combination of the probability distributions 232 that increases support for candidate answers consistently supported by the expert models 220 and moderates the influence of an expert model 220 whose probability distribution 232 diverges from the others. The log-space weighted sum 243 produces the combined probability distribution 244. A softmax 245 normalizes the combined probability distribution 244 to produce a normalized probability distribution 246 that assigns a normalized probability to individual candidate answers of the candidate-answer set 230. In some implementations, when the probability distributions 232 are mutually divergent, the softmax 245 yields a more uniform normalized probability distribution 246 than when the probability distributions 232 are mutually consistent, so that divergence among the expert models 220 flattens the normalized probability distribution 246 rather than overstating a contested candidate answer.

The normalized probability distribution 246 and the probability distributions 232, the latter being provided as ranked distributions 232, enter a cascade-boosting module 250. The cascade-boosting module 250 is configured to process rank-frequency information across the probability distributions 232. The objective of the cascade-boosting module 250 is to increase the probability of a candidate answer that appears within a threshold number of highest ranks in two or more of the probability distributions 232, relative to the probability of that candidate answer in the normalized probability distribution 246. The cascade-boosting module 250 constructs a rank-frequency table 252, which records, for individual candidate answers of the candidate-answer set 230, a frequency of the candidate answer at individual ranks across the ranked distributions 232. The cascade-boosting module 250 applies cascade weights 254, which are rank-dependent weights that decrease monotonically with rank. The cascade-boosting module 250 applies a boost scaler 256, which is a tunable parameter that adjusts the relative influence of the rank-frequency contribution. The cascade-boosting module 250 computes boosted scores 258, each boosted score being a sum of the normalized probability of a candidate answer and a product of the boost scaler 256 and a rank-weighted sum of the frequency of the candidate answer across the ranks. A softmax 259 re-normalizes the boosted scores 258 to produce the final probability distribution 260.

The final probability distribution 260, the rationales 234, and the specialties 214 enter a consensus model 270. The consensus model 270 is a model that is separate from the expert models 220 and is configured to emit the final answer 280 based at least in part on the final probability distribution 260 and on a respective rationale 234 received from individual expert models 220. The consensus model 270 provides an additional model-execution stage after probability weighting and cascade boosting, so that the final answer 280 is generated based on the final probability distribution 260 and the rationale outputs associated with the probability distributions 232. In some implementations the consensus model 270 emits the final answer 280 further based on a respective specialty 214 mapped to individual expert models 220 and on a strength of the respective rationale 234, and may select the final answer 280 based on the strength of the respective rationale 234 independently of which candidate answer has a highest probability in the final probability distribution 260.

The system 200 provides the final answer 280, together with a representation of the final probability distribution 260, as a decision support output 282. The decision support output 282 is delivered over one or more networks 206 to a computing device 204, which presents the decision support output 282 to a user. The decision support output 282 thereby includes both the final answer 280 and probability information associated with candidate answers. In this manner, the system 200 transforms the query 202 into the final answer 280 through the ordered operation of the triage model 210, the plurality of expert models 220, the probability-weighting module 240, the cascade-boosting module 250, and the consensus model 270.

### Probability-Weighting Module

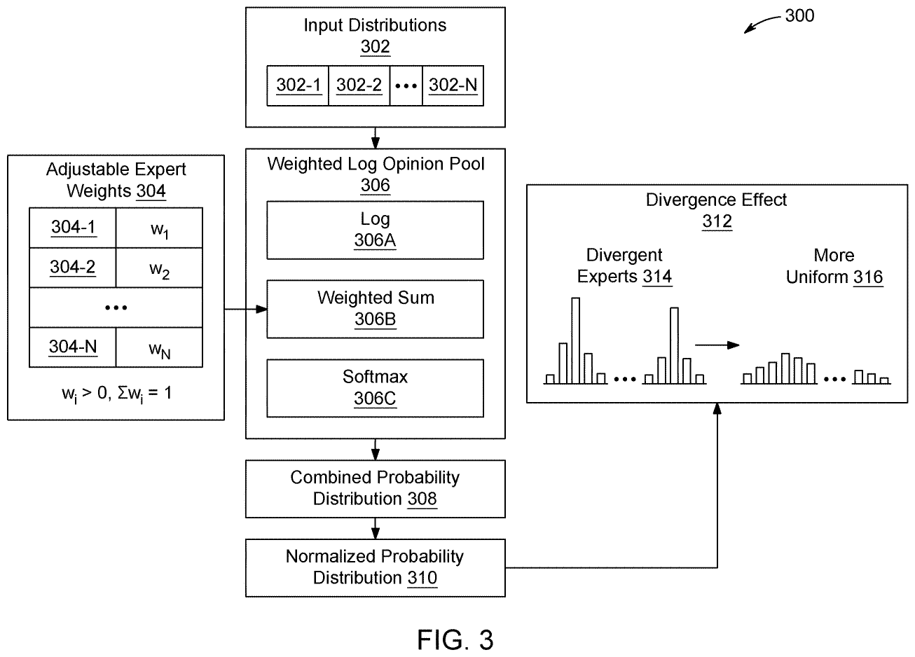

FIG. 3 illustrates a probability-weighting module 300 embodied as a weighted log opinion pool 306, which combines a plurality of input distributions 302 into a normalized probability distribution 310. The weighted log opinion pool 306 receives the input distributions 302, combines the input distributions 302 as a weighted sum in logarithmic space under a set of adjustable expert weights 304, and normalizes the result, thereby generating a single normalized probability distribution 310 over a set of candidate answers based on the plurality of input distributions 302.

The input distributions 302 are the probability distributions produced by a plurality of expert models, shown as input distributions 302-1, 302-2, through 302-N. Each input distribution 302 assigns, across the set of candidate answers, a probability to individual candidate answers, the probabilities assigned by an individual input distribution 302 summing to one. The objective of operating on the input distributions 302, rather than on a single answer from each expert model, is to preserve relative probability support assigned to each candidate answer by each expert model. Each input distribution 302 may be expressed, for example, as a vector of probability values indexed by candidate answer, as a set of probability values associated with named candidate answers, or in a similar form.

The weighted log opinion pool 306 applies the adjustable expert weights 304. The adjustable expert weights 304 are per-expert weights, shown as adjustable expert weights 304-1, 304-2, through 304-N and having values W₁, W₂, through W_{N}, that govern the contribution of each input distribution 302 to the combination. The adjustable expert weights 304 are constrained such that each weight is positive and the weights sum to one, expressed as wᵢ greater than zero and the sum of wᵢ equal to one. The objective of the adjustable expert weights 304 is to allow the contribution of an individual expert model to be increased or decreased according to a configured weighting parameter. In some implementations, the adjustable expert weights 304 are uniform across the input distributions 302, and in other implementations the adjustable expert weights 304 are assigned based on a measure of model reliability, a measure of model relevance to the task, or a similar model-specific measure.

The weighted log opinion pool 306 comprises a log 306A, a weighted sum 306B, and a softmax 306C. The log 306A is configured to apply a logarithmic transformation to individual input distributions 302, so that the subsequent combination is performed in logarithmic space. The weighted sum 306B is configured to compute, for individual candidate answers, a weighted sum of the logarithmically transformed input distributions 302 under the adjustable expert weights 304. Combining in logarithmic space under the adjustable expert weights 304 computes a weighted geometric combination of the input distributions 302, which increases probability support for a candidate answer that is consistently assigned probability support by the input distributions 302 and moderates influence of an input distribution 302 that diverges from the other input distributions 302. The weighted sum 306B produces a combined probability distribution 308, which is the distribution formed by the weighted sum in logarithmic space of the input distributions 302. The softmax 306C is configured to normalize the combined probability distribution 308 by applying a softmax function across the set of candidate answers, producing a normalized probability distribution 310 that assigns a normalized probability to individual candidate answers.

The weighted sum in logarithmic space computed by the weighted sum 306B is expressed, for individual candidate answers, as a combined probability distribution that sums, over the plurality of input distributions 302, a logarithm of the probability assigned to a candidate answer by an input distribution 302 weighted by the corresponding adjustable expert weight 304, in accordance with the following relation:

$${P}_{combined} ( X ) = {\sum }_{i} {w}_{i} log {p}_{i} ( X )$$

The softmax 306C normalizes the combined probability distribution by applying a softmax function across the set of candidate answers, expressed for individual candidate answers in accordance with the following relation:

$${P}_{normalized} ( X ) = \frac{{e}^{{P}_{combined} ( X )}}{{\sum }_{j} {e}^{{P}_{combined} ( {X}_{j} )}}$$

The weighted log opinion pool 306 exhibits a divergence effect 312, which characterizes the behavior of the normalized probability distribution 310 when the input distributions 302 diverge from one another. When the input distributions 302 are mutually divergent, shown as divergent experts 314, the normalization yields a normalized probability distribution 310 that is more uniform, shown as more uniform 316, than the normalized probability distribution 310 produced when the input distributions 302 are mutually consistent. The objective of the divergence effect 312 is to reflect divergence among the input distributions 302 in the normalized probability distribution 310, so that a contested candidate answer does not receive overstated probability support. The normalized probability distribution 310 produced by the weighted log opinion pool 306 thereby provides a probability-based combination of the input distributions 302, in which confidence indicated by the normalized probability distribution 310 reflects a degree of agreement among the input distributions 302.

### Cascade-Boosting Module

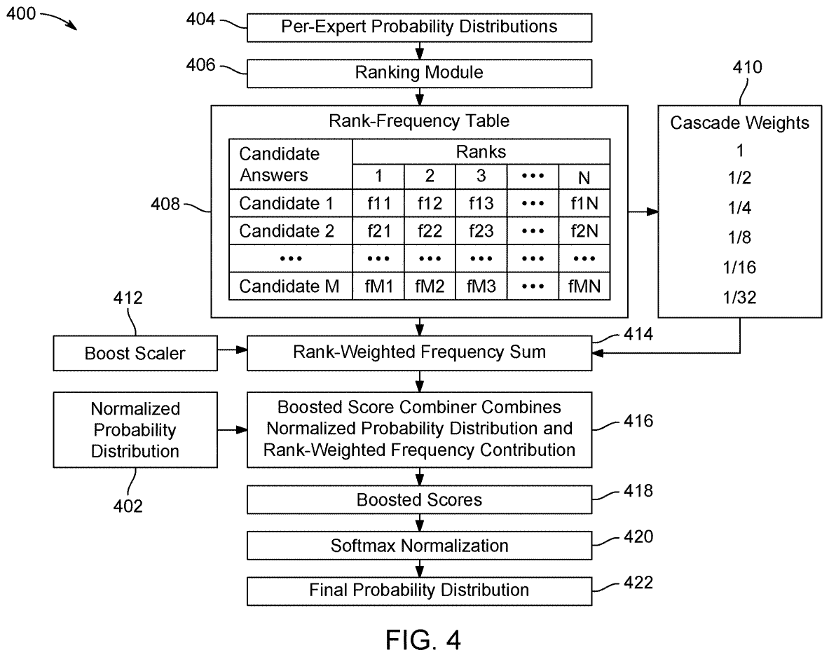

FIG. 4 illustrates a cascade-boosting module 400, which transforms a normalized probability distribution 402 into a final probability distribution 422 by reweighting candidate answers according to how frequently the candidate answers appear at high ranks across a plurality of per-expert probability distributions 404. The cascade-boosting module 400 identifies candidate-answer rank-frequency information across the plurality of per-expert probability distributions 404 and incorporates the candidate-answer rank-frequency information into the final probability distribution 422.

The normalized probability distribution 402 is the distribution received by the cascade-boosting module 400 as a starting point for boosting, and assigns a normalized probability to individual candidate answers of a set of candidate answers. The objective of receiving the normalized probability distribution 402 is to preserve the aggregated probability information generated upstream of the cascade-boosting module 400 as the base of the boosted result, so that the boosting modifies the aggregated probability information rather than replacing it. The normalized probability distribution 402 may be expressed, for example, as a vector of normalized probability values indexed by candidate answer, or in a similar form.

The per-expert probability distributions 404 are the probability distributions produced by individual expert models, each assigning a probability to individual candidate answers. The objective of providing the per-expert probability distributions 404 to the cascade-boosting module 400 is to make available the rank that each expert model assigns to each candidate answer, because the boosting depends on rank information that the normalized probability distribution 402 alone does not carry. The per-expert probability distributions 404 may comprise the same probability distributions that were combined to form the normalized probability distribution 402, taken here for the purpose of ranking.

The ranking module 406 is configured to determine, from individual per-expert probability distributions 404, a rank of individual candidate answers within that per-expert probability distribution 404. The objective of the ranking module 406 is to convert each per-expert probability distribution 404 into an ordered list of candidate answers, so that the frequency of each candidate answer at each rank can be tabulated. The ranking module 406 may rank the candidate answers, for example, in descending order of probability, such that the candidate answer with the highest probability in a per-expert probability distribution 404 is assigned the first rank, the candidate answer with the next highest probability is assigned the second rank, and so on.

The rank-frequency table 408 is the data structure that records, for individual candidate answers of the set of candidate answers, a frequency of the candidate answer at individual ranks of a plurality of ranks across the per-expert probability distributions 404. The rank-frequency table 408 is configured as an arrangement of candidate answers against ranks, in which a cell records the frequency, denoted fMN for a candidate answer M at a rank N, with which the candidate answer appears at that rank across the per-expert probability distributions 404. The objective of the rank-frequency table 408 is to capture, for each candidate answer, the pattern of ranks the candidate answer receives across the expert models, so that a candidate answer that recurs at high ranks is identifiable even when no single expert model places that candidate answer first. The rank-frequency table 408 may include a row for individual candidate answers, shown as candidate 1, candidate 2, through candidate M, and a column for individual ranks, shown as rank 1, rank 2, rank 3, through rank N, with the cells holding the frequencies f11, f12, f13 through f1N for the first candidate answer, f21, f22, f23 through f2N for the second candidate answer, and fM1, fM2, fM3 through fMN for the candidate answer M.

The cascade weights 410 are a set of rank-dependent weights that decrease monotonically with rank, configured such that the weight at the first rank is one and the weight at each successive rank is one-half of the weight at the immediately preceding rank, shown as the sequence 1, ½, ¼, ⅛, 1/16, and 1/32. The objective of the cascade weights 410 is to assign a greater rank-frequency contribution to a candidate answer that appears at a higher rank than to a candidate answer that appears at a lower rank. The sequence shown is exemplary, and in other implementations the cascade weights 410 follow a different monotonically decreasing schedule, provided that the weight at a given rank is not greater than the weight at any higher rank.

The cascade weights 410 are expressed, in an exemplary implementation, by a recurrence in which the weight at a first rank is one and the weight at each successive rank is one-half of the weight at the immediately preceding rank, yielding an exemplary sequence, in accordance with the following relations:

$${θ}_{r + 1} = \frac{1}{2} {θ}_{r}$$

$${θ}_{r} = [ 1. , 0.5 , 0.25 , 0.125 , 0.0625 , 0.03125 ]$$

The boost scaler 412 is a tunable parameter that scales the contribution of the rank-weighted frequency to the boosted score. The objective of the boost scaler 412 is to allow the relative influence of the boosting to be increased or decreased, so that a configuration of the cascade-boosting module 400 controls how strongly the rank-frequency information modifies the normalized probability distribution 402. The boost scaler 412 may be set, for example, to a small value to apply a mild boost that slightly adjusts the normalized probability distribution 402, or to a larger value to apply a stronger boost that more substantially raises a frequently high-ranked candidate answer, and the boost scaler 412 may be tuned based on a performance objective.

The rank-weighted frequency sum 414 is configured to compute, for individual candidate answers, a sum over the plurality of ranks of the frequency of the candidate answer at each rank weighted by the corresponding cascade weight 410. The objective of the rank-weighted frequency sum 414 is to reduce the row of the rank-frequency table 408 for a candidate answer to a single value that represents how often, and at how high a rank, the candidate answer appears across the expert models. The rank-weighted frequency sum 414 thereby produces, for a candidate answer, a value that is larger when the candidate answer appears frequently at high ranks and smaller when the candidate answer appears infrequently or only at low ranks.

The boosted score combiner 416 is configured to combine the normalized probability distribution 402 and the rank-weighted frequency contribution into boosted scores 418. The boosted score combiner 416 computes, for individual candidate answers, a boosted score as the sum of the normalized probability of the candidate answer and the product of the boost scaler 412 and the rank-weighted frequency sum 414 for the candidate answer. The objective of the boosted score combiner 416 is to adjust the aggregated probability information represented by the normalized probability distribution 402 by the rank-frequency information, so that the boosted scores 418 reflect both the aggregated probability and the recurrence of a candidate answer at high ranks.

The boosted score combiner 416 computes, for individual candidate answers, the boosted score 418 as the sum of the normalized probability of the candidate answer and the product of the boost scaler 412 and the rank-weighted frequency sum 414 of the candidate answer over the plurality of ranks, in accordance with the following relation:

$$BoostedScore ( X ) = {P}_{n o r m a l i z e d} ( X ) + {λ}_{b o o s t} \cdot {\sum }_{r} ( {f}_{X , r} \times {θ}_{r} )$$

The boosted scores 418 are the per-candidate values produced by the boosted score combiner 416. Each boosted score 418 is associated with a candidate answer and is larger for a candidate answer that has both a high normalized probability and a high rank-weighted frequency. The objective of the boosted scores 418 is to provide an intermediate quantity that ranks the candidate answers after boosting and before normalization, so that the boosted scores 418 can be converted into a probability distribution.

The softmax normalization 420 is configured to re-normalize the boosted scores 418 by applying a softmax function across the set of candidate answers, producing a final probability distribution 422. The objective of the softmax normalization 420 is to convert the boosted scores 418, which need not sum to one, into a probability distribution that assigns a probability to individual candidate answers and that sums to one. The final probability distribution 422 is the distribution produced by the cascade-boosting module 400, and increases the probability of a candidate answer that is ranked highly across two or more of the per-expert probability distributions 404 relative to the probability of that candidate answer in the normalized probability distribution 402. The final probability distribution 422 thereby reflects rank-frequency information derived from the per-expert probability distributions 404 in addition to the normalized probability distribution 402.

The softmax normalization 420 re-normalizes the boosted scores 418 by applying a softmax function across the set of candidate answers to produce the final probability distribution 422, expressed for individual candidate answers in accordance with the following relation:

$${P}_{f i n a l} ( X ) = \frac{{e}^{B o o s t e d S c o r e ( X )}}{{\sum }_{j} {e}^{B o o s t e dScore ( {X}_{j} )}}$$

### Consensus Model

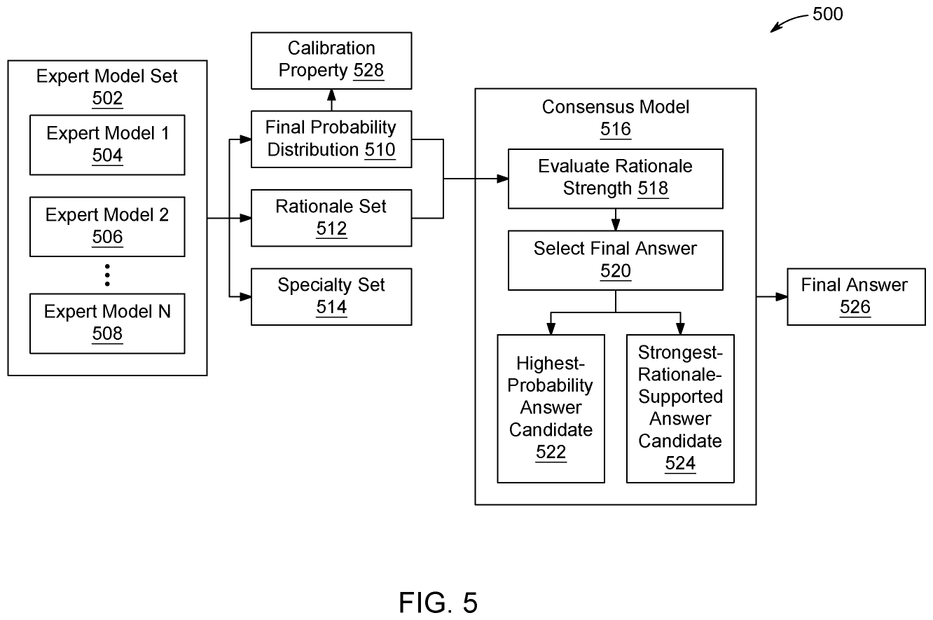

FIG. 5 illustrates an architecture 500 of a consensus model 516, which generates a final answer 526 from a final probability distribution 510, a rationale set 512, and a specialty set 514. The consensus model 516 is separate from an expert model set 502, and operates on the outputs of the expert model set 502 to produce the final answer 526 based on both the final probability distribution 510 and the rationales associated with candidate answers.

The expert model set 502 is the plurality of expert models whose outputs the consensus model 516 receives, shown as expert model 1 504, expert model 2 506, through expert model N 508. Each expert model in the expert model set 502 is an independently configured generative language model that processes a query and produces a probability distribution over a set of candidate answers and an associated rationale. The objective of depicting the expert model set 502 as separate from the consensus model 516 is to indicate that the consensus model 516 is a distinct model that operates on the outputs of the expert model set 502, rather than a component of any expert model in the expert model set 502. In some implementations the consensus model 516 and the expert models in the expert model set 502 are distinct models, and in some implementations the consensus model 516 is a distinct configuration of a model of a type also used in the expert model set 502.

The final probability distribution 510 is a first input to the consensus model 516. The final probability distribution 510 assigns a probability to individual candidate answers of the set of candidate answers and is produced by aggregating and reweighting the probability distributions of the expert model set 502. The objective of providing the final probability distribution 510 to the consensus model 516 is to make available aggregated probability support assigned to each candidate answer.

The rationale set 512 is a second input to the consensus model 516. The rationale set 512 comprises a respective rationale received from individual expert models of the expert model set 502, each rationale being a rationale output that an expert model produces in association with its probability distribution. The objective of providing the rationale set 512 to the consensus model 516 is to make available, for generation of the final answer 526, rationale information associated with the candidate answers in addition to aggregated probabilities. A rationale in the rationale set 512 may comprise, for example, clinical reasoning, scientific reasoning, elimination reasoning that indicates why particular candidate answers are excluded, or a similar reasoning output.

The specialty set 514 is a third input to the consensus model 516. The specialty set 514 comprises a respective specialty mapped to individual expert models of the expert model set 502. The objective of providing the specialty set 514 to the consensus model 516 is to make available the mapped specialty associated with each rationale, so that generation of the final answer 526 can be conditioned on the specialty from which a given rationale and probability distribution were produced. In some implementations the consensus model 516 uses the specialty set 514 to associate a rationale with the mapped specialty that produced the rationale, and in some implementations the consensus model 516 determines the final answer 526 without reference to the specialty set 514.

The consensus model 516 comprises an evaluate rationale strength 518 and a select final answer 520. The evaluate rationale strength 518 is configured to determine, for individual candidate answers, a rationale-strength measure indicating support that the rationale set 512 provides for the candidate answer. The objective of the evaluate rationale strength 518 is to characterize rationale strength associated with each candidate answer, so that generation of the final answer 526 can use the rationale-strength measure in addition to the final probability distribution 510. The rationale-strength measure may be derived, for example, from consistency of rationales associated with a candidate answer, specificity of those rationales, or a similar rationale characteristic.

The select final answer 520 is configured to determine the final answer 526 based at least in part on the final probability distribution 510 and on the rationale-strength measure determined by the evaluate rationale strength 518. The select final answer 520 may determine the final answer 526 as a highest-probability answer candidate 522, which is the candidate answer having a highest probability in the final probability distribution 510, or as a strongest-rationale-supported answer candidate 524, which is a candidate answer for which the rationale set 512 provides the greatest rationale-strength measure. The objective of providing for both the highest-probability answer candidate 522 and the strongest-rationale-supported answer candidate 524 is to indicate that the consensus model 516 is not restricted to returning the candidate answer with the highest probability in the final probability distribution 510, and in some implementations determines the final answer 526 to be the strongest-rationale-supported answer candidate 524 independently of which candidate answer has a highest probability in the final probability distribution 510. The final answer 526 is the output of the consensus model 516 and is the answer to the query that the consensus model 516 generates.

The final probability distribution 510 received by the consensus model 516 has a calibration property 528. The calibration property 528 is a correspondence between a confidence indicated by the final probability distribution 510 and an observed accuracy associated with the final probability distribution 510. In some implementations the calibration property 528 is such that the correspondence between the confidence indicated by the final probability distribution 510 and the observed accuracy is closer than the corresponding correspondence for a probability distribution produced by any single expert model of the expert model set 502. The objective of the calibration property 528 is to characterize the final probability distribution 510 as a distribution whose indicated confidence corresponds to observed accuracy, so that the final answer 526 and the final probability distribution 510 can be provided as probability-associated output data.

### Generation of a Final Answer to a Query

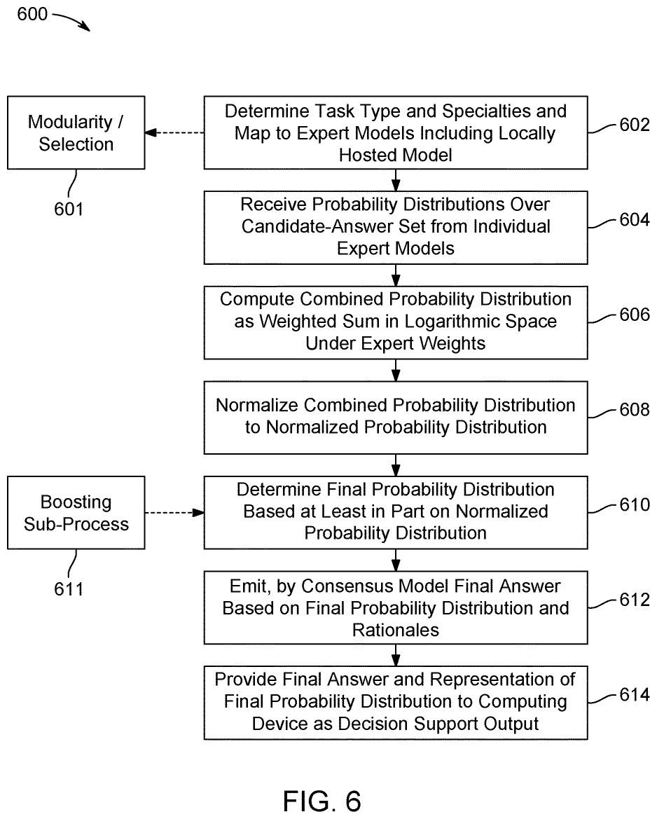

FIG. 6 illustrates a process 600 by which the system generates a final answer to a query. The process 600 routes the query to a plurality of expert models, obtains a probability distribution from individual expert models, aggregates the probability distributions, determines a final probability distribution, and produces the final answer through a consensus model that is separate from the expert models. The operations of the process 600 are performed by one or more processors executing instructions stored by one or more non-transitory computer-readable storage media.

At an operation 602, the process 600 determines, by a triage model and based on the query, a task type and a plurality of specialties, and maps individual specialties of the plurality of specialties to corresponding expert models of a plurality of expert models, the plurality of expert models including a locally hosted model. The objective of the operation 602 is to identify a task category associated with the query and to select corresponding expert models based on the plurality of specialties. Individual expert models comprise respective generative language models, and the locally hosted model processes the query within an infrastructure that retains the query, so that the query is not transmitted to a service external to the infrastructure. In some implementations the operation 602 adds an expert model to, or removes an expert model from, the plurality of expert models based on the task type, and a selection of the expert models is described in connection with a modularity and selection operation reached through a connector 601.

At an operation 604, the process 600 receives, from individual expert models of the plurality of expert models, a probability distribution over a candidate-answer set. The objective of the operation 604 is to obtain, from each expert model, probability values assigned to individual candidate answers as a distribution rather than as a single candidate answer. Individual expert models are independently configured to process the query according to a mapped specialty, and individual probability distributions assign, across the candidate-answer set, a probability to individual candidate answers.

At an operation 606, the process 600 computes a combined probability distribution as a weighted sum, in logarithmic space, of the probability distributions received from the plurality of expert models, under expert weights. The objective of the operation 606 is to combine the probability distributions into a single distribution that reflects aggregated probability support for individual candidate answers. The expert weights are positive and sum to one, and the weighted sum in logarithmic space computes a combination that increases probability support for a candidate answer consistently supported by the probability distributions and moderates the influence of a probability distribution that diverges from the others. In some implementations the expert weights are adjustable to vary the contribution of a corresponding expert model.

At an operation 608, the process 600 normalizes the combined probability distribution to produce a normalized probability distribution. The objective of the operation 608 is to convert the combined probability distribution into a distribution that assigns a normalized probability to individual candidate answers of the candidate-answer set. In some implementations the operation 608 applies a softmax function across the candidate-answer set.

At an operation 610, the process 600 determines a final probability distribution based at least in part on the normalized probability distribution. The objective of the operation 610 is to produce the final probability distribution used for generating the final answer. In some implementations the operation 610 determines the final probability distribution to be the normalized probability distribution, and in some implementations the operation 610 determines the final probability distribution by applying a boosting operation to the normalized probability distribution, the boosting operation being described in connection with a boosting sub-process reached through a connector 611.

At an operation 612, the process 600 emits, by a consensus model that is separate from the plurality of expert models, a final answer based at least in part on the final probability distribution and on a respective rationale received from individual expert models. The objective of the operation 612 is to generate the final answer through a model-execution stage that processes the final probability distribution and rationale data associated with the candidate answers. In some implementations the operation 612 emits the final answer further based on a respective specialty mapped to individual expert models and on a measure of support that the rationales provide for individual candidate answers, and determines the final answer independently of which candidate answer has a highest probability in the final probability distribution.

At an operation 614, the process 600 provides the final answer and a representation of the final probability distribution to a computing device as a decision support output. The objective of the operation 614 is to provide, to the computing device, output data including both the final answer and probability information associated with candidate answers. In some implementations the representation of the final probability distribution comprises two or more of the candidate answers ranked according to the final probability distribution. The process 600 thereby transforms the query into the final answer through the ordered operations of determining the task type and specialties, receiving the probability distributions, computing and normalizing the combined probability distribution, determining the final probability distribution, emitting the final answer, and providing the decision support output.

### Final Probability Distribution Determination

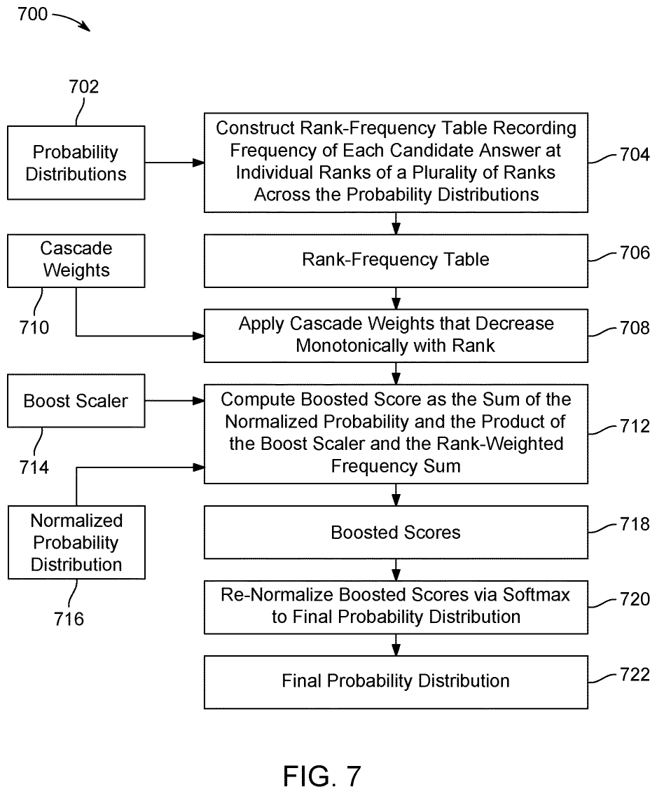

FIG. 7 illustrates a process 700 by which a final probability distribution 722 is determined from a normalized probability distribution 714 and probability distributions 710 using cascade boosting. The process 700 receives the probability distributions 710 as rank-source data, receives the normalized probability distribution 714 as base probability data, and produces the final probability distribution 722. The process 700 reweights candidate answers according to frequencies with which the candidate answers appear at ranks across the probability distributions 710, such that a candidate answer ranked within a threshold number of highest ranks in two or more of the probability distributions 710 can receive an increased probability relative to a probability of the candidate answer in the normalized probability distribution 714.

At an operation 702, the process 700 constructs a rank-frequency table 706 recording a frequency of individual candidate answers at individual ranks of a plurality of ranks across the probability distributions 710. The objective of the operation 702 is to capture, for individual candidate answers, rank-position data across the probability distributions 710. The probability distributions 710 are the probability distributions produced by the plurality of expert models and are used by the process 700 for ranking the candidate answers. The rank-frequency table 706 is the data structure that holds, for individual candidate answers, the frequency with which the candidate answer appears at individual ranks across the probability distributions 710. In some implementations, the rank-frequency table 706 is arranged as candidate answers against ranks, with a cell recording the frequency of a candidate answer at a corresponding rank.

At an operation 704, the process 700 applies cascade weights 712 that decrease monotonically with rank. The objective of the operation 704 is to assign a greater rank-frequency contribution to a candidate answer that appears at a higher rank than to a candidate answer that appears at a lower rank. The cascade weights 712 are rank-dependent weights configured such that the weight at a given rank is not greater than the weight at any higher rank. In some implementations, the cascade weights 712 are configured such that the weight at a first rank is one and the weight at each successive rank is one-half of the weight at the immediately preceding rank. In other implementations, the cascade weights 712 follow a different monotonically decreasing schedule.

At an operation 708, the process 700 computes a boosted score as a sum of a normalized probability and a product of a boost scaler 716 and a rank-weighted frequency sum, producing boosted scores 718. The objective of the operation 708 is to modify the normalized probability distribution 714 using rank-frequency information, so that the boosted scores 718 reflect both the normalized probability of a candidate answer and the recurrence of that candidate answer at high ranks. The normalized probability distribution 714 is the distribution received as the base of the boosting operation and assigns a normalized probability to individual candidate answers. The boost scaler 716 is a tunable parameter that scales the contribution of the rank-weighted frequency sum to the boosted score. In some implementations, the boost scaler 716 is set to a smaller value to apply a lower boosting contribution or to a larger value to apply a higher boosting contribution. The rank-weighted frequency sum is computed, for individual candidate answers, as a sum over the plurality of ranks of the frequency of the candidate answer at each rank weighted by the corresponding cascade weight 712. The boosted scores 718 are the per-candidate values produced by the operation 708, with each boosted score being larger for a candidate answer that has both a high normalized probability and a high rank-weighted frequency.

At an operation 720, the process 700 re-normalizes the boosted scores 718 to produce the final probability distribution 722. The objective of the operation 720 is to convert the boosted scores 718, which need not sum to one, into a distribution that assigns a probability to individual candidate answers and that sums to one. In some implementations, the operation 720 applies a softmax function across the candidate answers. The final probability distribution 722 is the distribution produced by the process 700 and increases the probability of a candidate answer that is ranked highly across two or more of the probability distributions 710 relative to the probability of that candidate answer in the normalized probability distribution 714. The process 700 provides the final probability distribution 722 to the operation that emits the final answer.

### Expert Model Selection

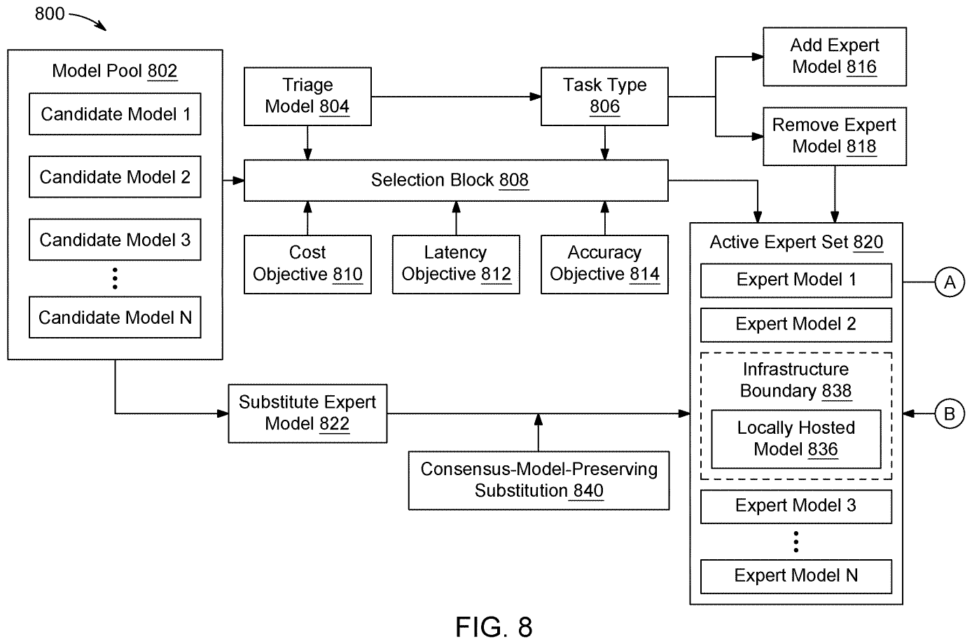

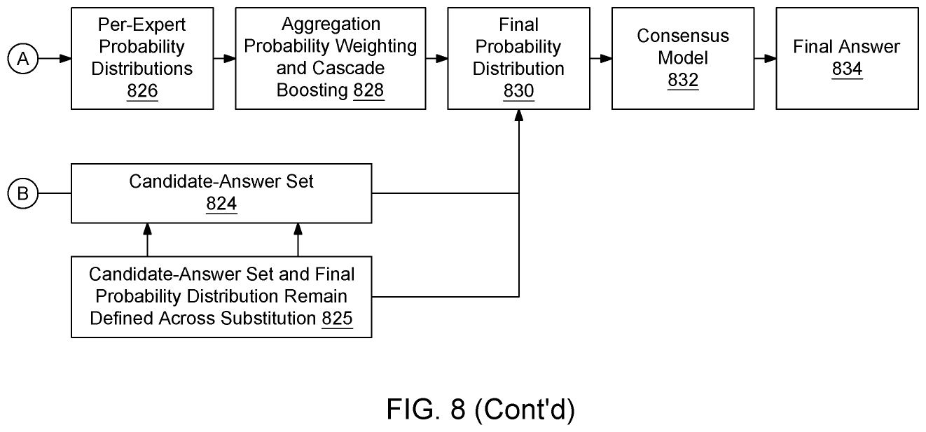

FIG. 8 illustrates an embodiment 800 in which a plurality of expert models is selected from a model pool 802 and is modified, so that the set of expert models used to process a query is configurable and substitutable without reconfiguration of a consensus model 832. The embodiment 800 selects the expert models according to a configurable objective, adds or removes an expert model based on a task type 806, and substitutes one expert model for a different model while preserving the operation of the consensus model 832.

The model pool 802 is a set of candidate models from which expert models are selected, shown as candidate model 1, candidate model 2, candidate model 3, through candidate model N. Each candidate model is a generative language model that is available to be selected as an expert model. The objective of the model pool 802 is to provide a set of available models that is larger than the set of expert models used for a given query, so that the set of expert models can be assembled and modified by selection from the model pool 802 rather than by reconfiguration of the system. The candidate models in the model pool 802 may comprise, for example, publicly available models, open-source models, models fine-tuned for particular specialties, models of differing computational cost, or a combination thereof, and the model pool 802 may be extended by adding a new candidate model.

The triage model 804 determines a task type 806 based on the query and provides the task type 806 to a selection block 808. The selection block 808 is configured to select, from the model pool 802, the expert models that populate an active expert set 820. The selection block 808 selects the expert models according to a configurable objective, shown as a cost objective 810, a latency objective 812, and an accuracy objective 814. The cost objective 810 is an objective to reduce a computational cost of processing the query. The latency objective 812 is an objective to reduce a time to produce the final answer 834. The accuracy objective 814 is an objective to increase an accuracy metric associated with the final answer 834. The objective of selecting according to the configurable objective is to allow the active expert set 820 to be assembled according to a configured system objective, such that a selection favoring the cost objective 810 selects candidate models of lower computational cost, a selection favoring the latency objective 812 selects candidate models associated with lower processing latency, and a selection favoring the accuracy objective 814 selects candidate models associated with a higher accuracy metric. In some implementations the selection block 808 selects the expert models according to one of the configurable objectives, and in some implementations the selection block 808 selects the expert models according to a combination of the configurable objectives.

The embodiment 800 modifies the active expert set 820 based on the task type 806. An add expert model 816 is an operation that adds an expert model to the active expert set 820, and a remove expert model 818 is an operation that removes an expert model from the active expert set 820. The objective of the add expert model 816 and the remove expert model 818 is to configure the active expert set 820 according to the task type 806, so that an expert model corresponding to the task type 806 is included and an expert model not corresponding to the task type 806 is excluded. In some implementations the add expert model 816 and the remove expert model 818 are performed based on the task type 806 determined by the triage model 804.

The active expert set 820 is the set of expert models selected to process the query, shown as expert model 1, expert model 2, expert model 3, through expert model N. The active expert set 820 includes at least one locally hosted model 836, which executes within an infrastructure delimited by an infrastructure boundary 838 and processes the query within the infrastructure that the infrastructure boundary 838 delimits, so that the query provided to the locally hosted model 836 is not transmitted to a service external to the infrastructure. The objective of including the locally hosted model 836 within the active expert set 820 is to enable processing of the query within the infrastructure without transmitting the query to a service external to the infrastructure.

The embodiment 800 substitutes one expert model of the active expert set 820 for a different model. A substitute expert model 822 is a model selected from the model pool 802 to replace an expert model of the active expert set 820. The substitution is performed as a consensus-model-preserving substitution 840, which is a substitution that replaces an expert model without reconfiguration of the consensus model 832. The objective of the consensus-model-preserving substitution 840 is to allow an expert model to be replaced, for example where a model becomes unavailable or where a different model satisfies the configurable objective, without modifying the consensus model 832 that produces the final answer 834. Across the substitution, the candidate-answer set 824 and the final probability distribution 830 remain defined, shown as the property 825, such that the substitution changes which expert models contribute to the active expert set 820 while the set of candidate answers over which the expert models operate and the form of the final probability distribution 830 are unchanged.

The active expert set 820 processes the query over the candidate-answer set 824 and produces per-expert probability distributions 826. The candidate-answer set 824 is the shared set of candidate answers over which individual expert models of the active expert set 820 define the per-expert probability distributions 826. The per-expert probability distributions 826 are provided to an aggregation 828, which performs probability weighting and cascade boosting to produce a final probability distribution 830. The objective of the aggregation 828 is to combine the per-expert probability distributions 826 into a single distribution and to reweight candidate answers according to their recurrence at high ranks, so that the final probability distribution 830 reflects aggregated probability information derived from the active expert set 820. The final probability distribution 830 is provided to the consensus model 832, which is separate from the expert models of the active expert set 820 and which emits a final answer 834 based at least in part on the final probability distribution 830 and on a rationale received from individual expert models of the active expert set 820. The embodiment 800 thereby produces the final answer 834 from an active expert set 820 that is selected according to the configurable objective, modified based on the task type 806, and substitutable without reconfiguration of the consensus model 832.

### Server Hardware Architecture

FIG. 9 illustrates a server hardware architecture 900 that implements the system. The server hardware architecture 900 comprises a server 901 that executes instructions which cause the system to route a query to a plurality of expert models, aggregate probability distributions produced by the expert models, and produce a final answer through a consensus model that is separate from the expert models. The server hardware architecture 900 provides physical and logical computing resources on which the models and modules of the system are stored and executed.

The server 901 comprises one or more processors 902. The one or more processors 902 are configured to execute instructions stored by a memory 904, thereby performing operations attributed to the system. The objective of the one or more processors 902 is to carry out routing, probability aggregation, boosting, and consensus-model execution operations of the system. The one or more processors 902 may comprise, for example, a central processing unit, a graphics processing unit, a tensor processing unit, a digital signal processor, a field-programmable gate array, an application-specific integrated circuit, a system-on-chip, or a combination thereof. In some implementations the one or more processors 902 comprise a plurality of processors that execute the expert models in parallel, which reduces a time required to produce the final answer.

The server 901 further comprises the memory 904. The memory 904 comprises one or more computer-readable storage media configured to store instructions executed by the one or more processors 902 and to store data objects produced and consumed by the system. In some implementations, the memory 904 comprises one or more non-transitory computer-readable storage media. The objective of the memory 904 is to hold the models, modules, and data of the system in a form accessible to the one or more processors 902. The memory 904 may comprise, for example, random access memory, read-only memory, electrically erasable programmable read-only memory, flash memory, a solid-state drive, a hard disk drive, an optical storage medium, a magnetic storage device, or a combination thereof, and in some implementations is configured as distributed storage across a plurality of machines. As used herein, a model or a module refers to a configured component implemented as instructions stored by the memory 904 and executed by the one or more processors 902, and a reference to a model or a module is not intended to invoke 35 U.S.C. § 112(f).

The memory 904 stores triage model instructions 924, expert model instructions 926, probability-weighting module instructions 928, cascade-boosting module instructions 930, and consensus model instructions 932. The triage model instructions 924 are instructions that, when executed, cause the system to determine a task type and a plurality of specialties from the query and to map the specialties to corresponding expert models. The expert model instructions 926 are instructions that, when executed, cause the plurality of expert models to process the query and to produce a probability distribution and a rationale. The probability-weighting module instructions 928 are instructions that, when executed, cause the system to compute a combined probability distribution as a weighted sum in logarithmic space of the probability distributions and to normalize the combined probability distribution. The cascade-boosting module instructions 930 are instructions that, when executed, cause the system to construct a rank-frequency table, apply cascade weights and a boost scaler, and produce a final probability distribution. The consensus model instructions 932 are instructions that, when executed, cause the system to emit the final answer based on the final probability distribution and the rationales. The objective of storing these instructions in the memory 904 is to make the models and modules of the system executable by the one or more processors 902 as configured components of the server 901.

The memory 904 further stores candidate-answer set data 934, expert weight data 936, cascade weight data 938, boost scaler data 940, and final probability distribution data 942. The candidate-answer set data 934 represents the set of candidate answers over which the expert models define the probability distributions. The expert weight data 936 represents the per-expert weights that are positive and sum to one and that are applied in the weighted sum in logarithmic space. The cascade weight data 938 represents the rank-dependent weights that decrease monotonically with rank. The boost scaler data 940 represents the tunable parameter that scales the rank-weighted frequency contribution. The final probability distribution data 942 represents the distribution used by the system to generate the final answer. The objective of storing these data objects in the memory 904 is to make the parameters, intermediate data, and output data of the system available to the one or more processors 902 during execution.

The server 901 further comprises one or more communication interfaces 906, a display device 908, one or more other I/O devices 910, and one or more mass storage devices 912, interconnected with the one or more processors 902 and the memory 904 by a bus 914. The one or more communication interfaces 906 are configured to enable the server 901 to communicate over one or more networks 916, and may support, for example, a wired network interface, a wireless network interface, or a combination thereof. The display device 908 is configured to present information from the server 901, and may comprise, for example, a monitor or a similar display. The one or more other I/O devices 910 are configured to provide input to and output from the server 901, and may comprise, for example, a keyboard, a pointing device, or a similar device. The one or more mass storage devices 912 are configured to provide persistent storage for the server 901, and may comprise, for example, a hard disk drive, a solid-state drive, or a similar device. The bus 914 is configured to carry data and control signals among the components of the server 901.

The server 901 communicates over the one or more networks 916 with a computing device 918. The one or more networks 916 comprise one or more wired networks, one or more wireless networks, or a combination thereof. The computing device 918 comprises an interface 920 configured to receive a decision support output 922 from the server 901 and to present the decision support output 922. The decision support output 922 comprises the final answer and a representation of the final probability distribution. The objective of the computing device 918 is to receive and present the decision support output 922 as output data generated by the server 901. The computing device 918 may comprise, for example, a laptop computer, a desktop computer, a tablet computer, a smartphone, a workstation, or a similar device. In this manner, the server hardware architecture 900 stores and executes the models and modules of the system and delivers the decision support output 922 to the computing device 918.

### Hardware and Inference Logic Substrate

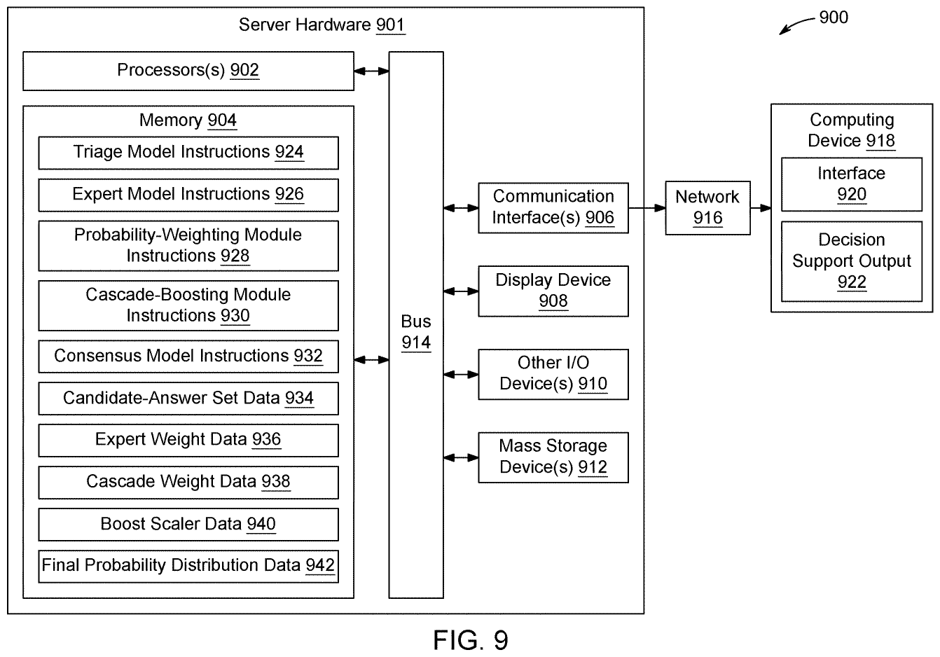

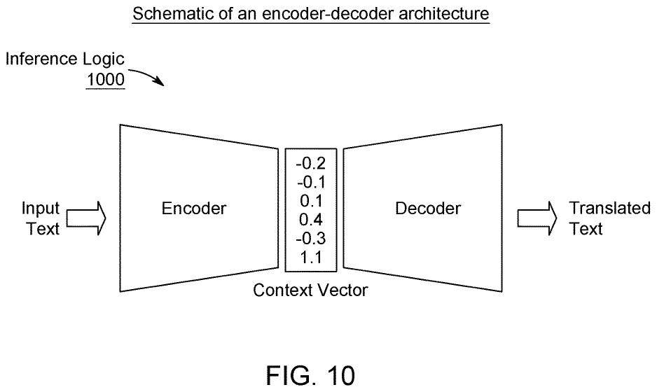

The consensus reasoning system described above with reference to FIG. 1 through FIG. 9 is implemented as a composition of software and hardware that operates upon concrete computing infrastructure and concrete artificial neural network inference logic. The cooperating models and modules of the system, comprising the triage model, the plurality of expert models, the probability-weighting module, the cascade-boosting module, and the consensus model, are not abstractions performed by mental steps and are not implementable through pen and paper methods. The system requires the computing hardware described above with reference to FIG. 9, and the artificial neural network inference logic described below with reference to FIG. 10 and the following figures, in order to perform the operations described above. A person cannot, in the mind or with pen and paper, obtain a probability distribution over a set of candidate answers from a generative language model, combine a plurality of such distributions as a weighted sum in logarithmic space, construct a rank-frequency table across the distributions, reweight the distributions by a tunable boost scaler, and emit a final answer through a separate model, at the scale and within the time required to answer a query.

Each of the cooperating models and modules depends upon the underlying computing hardware in a specific and necessary manner. The triage model 210 receives the query 202 and determines the task type 212 and the plurality of specialties 214 through inference logic executed by the one or more processors 902 of FIG. 9. The plurality of expert models 220 process the query 202 and produce the probability distributions 232 and the rationales 234 through generative language model inference executed by the one or more processors 902, the one or more processors 902 in some implementations comprising a plurality of processors that execute the expert models 220 in parallel to produce the probability distributions 232 within the time required to answer the query 202. The probability-weighting module 240 computes the combined probability distribution 244 as the weighted sum in logarithmic space and normalizes the combined probability distribution 244 to the normalized probability distribution 246 through numerical operations executed by the one or more processors 902 upon data held in the memory 904. The cascade-boosting module 250 constructs the rank-frequency table 252, applies the cascade weights 254 and the boost scaler 256, and produces the final probability distribution 260 through numerical operations executed by the one or more processors 902 upon data held in the memory 904. The consensus model 270 emits the final answer 280 through inference logic executed by the one or more processors 902. Each of the foregoing operations is performed by the one or more processors 902, the memory 904, and the one or more communication interfaces 906 of FIG. 9, and none is realizable by software described in the abstract or by a person reasoning unaided.

The system further depends upon the artificial neural network inference logic of FIG. 10 and the following figures in a specific and necessary manner. The triage model 210, the expert models 220, and the consensus model 270 are each realized as a generative language model that performs inference through the artificial neural network architectures described with reference to FIG. 10 and the following figures, including one or more of an encoder-decoder architecture, an attention mechanism, a self-attention computation, a multi-head attention computation, a transformer encoder, a transformer decoder, and a vision transformer. The determination of the task type 212 and the plurality of specialties 214 by the triage model 210 depends upon the inference logic of an attention-based language model that processes the query 202 as a sequence of tokens. The production of the probability distributions 232 by the expert models 220 depends upon the inference logic of a transformer-based language model that, for a query 202 processed according to a mapped specialty, produces a probability over individual candidate answers of the candidate-answer set 230. The emission of the final answer 280 by the consensus model 270 depends upon the inference logic of a transformer-based language model that processes the final probability distribution 260 and the rationales 234. The inference logic is realized as concrete computer-readable program instructions executed by the one or more processors 902 of FIG. 9 and is not realized in human cognition. FIG. 10 and the following figures describe representative artificial neural network architectures by which the triage model 210, the expert models 220, and the consensus model 270 are implemented, and these architectures are provided as examples of the inference logic upon which the system depends rather than as limitations on the form of the models.

Together, the computing hardware of FIG. 9 and the artificial neural network inference logic of FIG. 10 and the following figures furnish the technical substrate upon which the consensus reasoning system operates. The system thereby addresses concrete technical problems rooted in computer technology, including the dependence of a conventional system upon a single generative language model whose revision, replacement, or repricing breaks the workflow built upon it, the tendency of a single generative language model to report a confidence that does not correspond to its observed accuracy, the inability of a conventional system to combine the full probability distributions of a plurality of generative language models in logarithmic space and to reweight the combination by a rank-frequency boosting transform, and the requirement of a conventional system to transmit a query containing sensitive information to a service external to the operator infrastructure. The technical advantages thereby achieved, including the production of a final probability distribution 260 whose indicated confidence corresponds to observed accuracy more closely than a probability distribution of any single expert model, the substitution of one expert model 220 for a different model without reconfiguration of the consensus model 270 while the candidate-answer set 230 and the final probability distribution 260 remain defined, and the processing of the query 202 by a locally hosted model 222 within an infrastructure that retains the query 202, arise only when the system operates upon the computing hardware and the artificial neural network inference logic described herein. The operations of the system are accordingly directed to a specific improvement in the manner in which a plurality of generative language models is coordinated by a computing system, and are not directed to a result divorced from the means of achieving it.

### Working Example 1

The following describes one example of the operation of the system 200, and is provided to illustrate the operations described above and not to limit the system 200 to the particular data of the example.

In this example, the system 200 is deployed within a hospital network as a clinical decision support tool, and operates within a local infrastructure under the control of the hospital. A query 202 is received that describes a patient who presents four days after a craniotomy with a swollen, warm, and tender calf, a low-grade fever, and a history of end-stage renal disease requiring dialysis, and that requests a most appropriate next step selected from a set of candidate answers 230. The set of candidate answers 230 in this example comprises ten mutually exclusive options, including a surgical thrombectomy, an inferior vena cava filter, an unfractionated heparin therapy, a direct oral anticoagulant therapy, and a low molecular weight heparin therapy, among others.

The triage model 210 processes the query 202 and determines a task type 212 of a treatment task and a plurality of specialties 214, the specialties 214 comprising hematology, nephrology, vascular surgery, and infectious disease. The triage model 210 performs the specialty mapping 236, which maps the hematology specialty to a first expert model 220-1, the nephrology specialty to a second expert model 220-2, the vascular surgery specialty to a third expert model, and the infectious disease specialty to a fourth expert model. In this example the second expert model 220-2 is a locally hosted model 222 that executes within the infrastructure delimited by the infrastructure boundary 224, so that the query 202, which contains patient information, is processed without transmission to a service external to the infrastructure.

Each expert model 220 processes the query 202 according to its mapped specialty and returns a probability distribution 232 over the set of candidate answers 230 and a rationale 234. In this example the first expert model 220-1 returns a probability distribution 232-1 that assigns a probability of 0.55 to the inferior vena cava filter, a probability of 0.25 to the unfractionated heparin therapy, and smaller probabilities to the remaining candidate answers, together with a rationale 234-1 that identifies a recent intracranial surgery as a contraindication to systemic anticoagulation. The second expert model 220-2 returns a probability distribution 232-2 that assigns a probability of 0.45 to the unfractionated heparin therapy and a probability of 0.35 to the inferior vena cava filter, together with a rationale 234-2 that identifies the reversibility and non-renal clearance of the unfractionated heparin therapy as favorable in end-stage renal disease. The third expert model returns a probability distribution that assigns its highest probability to the low molecular weight heparin therapy, together with a rationale favoring anticoagulation. The fourth expert model returns a probability distribution that assigns a probability of 0.55 to the inferior vena cava filter, together with a rationale that attributes the inflammatory signs to thrombosis rather than infection.

The probability-weighting module 240 combines the probability distributions 232 into a combined probability distribution 244 by the log-space weighted sum 243, under expert weights 242 that are positive and sum to one. In this example the expert weights 242 are assigned such that the hematology and vascular surgery specialties, which the task type 212 identifies as most relevant, contribute more than the remaining specialties. Because the expert models 220 diverge, with one expert model favoring the low molecular weight heparin therapy that the other expert models disfavor, the softmax 245 produces a normalized probability distribution 246 that is more uniform than it would be were the expert models 220 in agreement, and the divergent low molecular weight heparin therapy is moderated rather than allowed to dominate.

The cascade-boosting module 250 then constructs the rank-frequency table 252 from the ranked distributions 232. In this example the inferior vena cava filter appears at the first rank in two of the probability distributions 232 and at the second rank in a third probability distribution 232, while the unfractionated heparin therapy appears at the first rank in one probability distribution 232 and at the second rank in two others. The cascade-boosting module 250 applies the cascade weights 254, in which the first rank weighs one, the second rank weighs one-half, and each lower rank weighs one-half of the rank above it, and scales the rank-weighted contribution by the boost scaler 256. The cascade-boosting module 250 adds the scaled contribution to the normalized probability of each candidate answer to form the boosted scores 258, and the softmax 259 re-normalizes the boosted scores 258 into the final probability distribution 260. In this example the boosting raises the inferior vena cava filter, which several expert models 220 rank highly, above the unfractionated heparin therapy in the final probability distribution 260.

The consensus model 270, which is separate from the expert models 220, receives the final probability distribution 260, the rationales 234, and the specialties 214. In this example the consensus model 270 determines that the rationale 234 associated with the inferior vena cava filter, which identifies an absolute contraindication to systemic anticoagulation in the recent post-craniotomy period, provides a stronger measure of support than the rationale associated with the unfractionated heparin therapy, and emits a final answer 280 of the inferior vena cava filter. In this example the final answer 280 coincides with the candidate answer having the highest probability in the final probability distribution 260, while in other examples the consensus model 270 emits a final answer 280 supported by the strongest rationale independently of which candidate answer has the highest probability.

The system 200 provides the final answer 280, together with a representation of the final probability distribution 260, to the computing device 204 as the decision support output 282. In this example the representation of the final probability distribution 260 comprises the candidate answers ranked according to the final probability distribution 260, with the inferior vena cava filter shown as most probable, the unfractionated heparin therapy shown as a next most probable alternative, and the remaining candidate answers shown with their respective probabilities. A clinician at the computing device 204 thereby receives both the final answer 280 and the confidence the system 200 associates with the candidate answers, which supports the clinician in reaching a determination.

In a further illustration of the modularity of the system 200, the hospital later replaces the third expert model with a different generative language model that the hospital regards as more accurate for vascular surgery. The system 200 substitutes the third expert model for the different model without reconfiguration of the consensus model 270, and the set of candidate answers 230 and the form of the final probability distribution 260 remain defined across the substitution. The system 200 thereafter answers a subsequent query of the same task type 212 using the substituted expert model, without a rebuild of the system 200. In another illustration, the hospital reconfigures the system 200 for a latency objective by selecting, for a time-sensitive query, expert models 220 that produce a result more quickly, and reconfigures the system 200 for an accuracy objective for a complex query by selecting expert models 220 associated with higher correctness.

Although this example is described in a clinical setting, the system 200 operates in the same manner in other settings. In one such setting, the query 202 describes a software system incident and the set of candidate answers 230 comprises candidate remediation actions, the specialties 214 comprise areas such as networking, storage, and security, and the final answer 280 identifies a remediation action. In another such setting, the query 202 describes a financial instrument and the set of candidate answers 230 comprises candidate classifications, the specialties 214 comprise areas of financial analysis, and the final answer 280 identifies a classification. In each setting the system 200 routes the query 202 to the plurality of expert models 220, combines the probability distributions 232 in logarithmic space, reweights the result by the cascade-boosting module 250, and emits the final answer 280 through the consensus model 270.

### Artificial Intelligence System Implementation

Some implementations of the technology disclosed relate to using a Transformer model to provide an AI system. In particular, the technology disclosed proposes a parallel input, parallel output (PIPO) AI system based on the Transformer architecture. The Transformer model relies on a self-attention mechanism to compute a series of context-informed vector-space representations of elements in the input sequence and the output sequence, which are then used to predict distributions over subsequent elements as the model predicts the output sequence element-by-element. Not only is this mechanism straightforward to parallelize, but as each input's representation is also directly informed by all other inputs' representations, this results in an effectively global receptive field across the whole input sequence. This stands in contrast to, e.g., convolutional architectures which typically only have a limited receptive field.

In one implementation, the disclosed AI system is a multilayer perceptron (MLP). In another implementation, the disclosed AI system is a feedforward neural network. In yet another implementation, the disclosed AI system is a fully connected neural network. In a further implementation, the disclosed AI system is a fully convolution neural network. In a yet further implementation, the disclosed AI system is a semantic segmentation neural network. In a yet another further implementation, the disclosed AI system is a generative adversarial network (GAN) (e.g., CycleGAN, StyleGAN, pixelRNN, text-2-image, DiscoGAN, IsGAN). In a yet another implementation, the disclosed AI system includes self-attention mechanisms like Transformer, Vision Transformer (ViT), Bidirectional Transformer (BERT), Detection Transformer (DETR), Deformable DETR, UP-DETR, DeiT, Swin, GPT, iGPT, GPT-2, GPT-3, various ChatGPT versions, various LLAMA versions, BERT, SpanBERT, ROBERTa, XLNet, ELECTRA, UniLM, BART, T5, ERNIE (THU), KnowBERT, DeiT-Ti, DeiT-S, DeiT-B, T2T-ViT-14, T2T-ViT-19, T2T-ViT-24, PVT-Small, PVT-Medium, PVT-Large, TNT-S, TNT-B, CPVT-S, CPVT-S-GAP, CPVT-B, Swin-T, Swin-S, Swin-B, Twins-SVT-S, Twins-SVT-B, Twins-SVT-L, Shuffle-T, Shuffle-S, Shuffle-B, XCIT-S12/16, CMT-S, CMT-B, VOLO-D1, VOLO-D2, VOLO-D3, VOLO-D4, MoCo v3, ACT, TSP, Max-DeepLab, VisTR, SETR, Hand-Transformer, HOT-Net, METRO, Image Transformer, Taming transformer, TransGAN, IPT, TTSR, STTN, Masked Transformer, CLIP, DALL-E, Cogview, UniT, ASH, TinyBert, FullyQT, ConvBert, FCOS, Faster R-CNN+FPN, DETR-DC5, TSP-FCOS, TSP-RCNN, ACT+MKDD (L=32), ACT+MKDD (L=16), SMCA, Efficient DETR, UP-DETR, UP-DETR, VITB/16-FRCNN, VIT-B/16-FRCNN, PVT-Small+RetinaNet, Swin-T+RetinaNet, Swin-T+ATSS, PVT-Small+DETR, TNT-S+DETR, YOLOS-Ti, YOLOS-S, and YOLOS-B.

In one implementation, the disclosed AI system is a convolution neural network (CNN) with a plurality of convolution layers. In another implementation, the disclosed AI system is a recurrent neural network (RNN) such as a long short-term memory network (LSTM), bi-directional LSTM (Bi-LSTM), or a gated recurrent unit (GRU). In yet another implementation, the disclosed AI system includes both a CNN and an RNN.

In yet other implementations, the disclosed AI system can use 1D convolutions, 2D convolutions, 3D convolutions, 4D convolutions, 5D convolutions, dilated or atrous convolutions, transpose convolutions, depthwise separable convolutions, pointwise convolutions, 1×1 convolutions, group convolutions, flattened convolutions, spatial and cross-channel convolutions, shuffled grouped convolutions, spatial separable convolutions, and deconvolutions. The disclosed AI system can use one or more loss functions such as logistic regression/log loss, multi-class cross-entropy/softmax loss, binary cross-entropy loss, mean-squared error loss, L1 loss, L2 loss, smooth L1 loss, and Huber loss. The disclosed AI system can use any parallelism, efficiency, and compression schemes such TFRecords, compressed encoding (e.g., PNG), sharding, parallel calls for map transformation, batching, prefetching, model parallelism, data parallelism, and synchronous/asynchronous stochastic gradient descent (SGD). The disclosed AI system can include upsampling layers, downsampling layers, recurrent connections, gates and gated memory units (like an LSTM or GRU), residual blocks, residual connections, highway connections, skip connections, peephole connections, activation functions (e.g., non-linear transformation functions like rectifying linear unit (ReLU), leaky ReLU, exponential liner unit (ELU), sigmoid and hyperbolic tangent (tanh)), batch normalization layers, regularization layers, dropout, pooling layers (e.g., max or average pooling), global average pooling layers, and attention mechanisms.

The disclosed AI system can be a linear regression model, a logistic regression model, an Elastic Net model, a support vector machine (SVM), a random forest (RF), a decision tree, and a boosted decision tree (e.g., XGBoost), or some other tree-based logic (e.g., metric trees, kd-trees, R-trees, universal B-trees, X-trees, ball trees, locality sensitive hashes, and inverted indexes). The disclosed AI system can be an ensemble of multiple models, in some implementations.

In some implementations, the disclosed AI system can be trained using backpropagation-based gradient update techniques. Example gradient descent techniques that can be used for training the disclosed AI system include stochastic gradient descent, batch gradient descent, and mini-batch gradient descent. Some examples of gradient descent optimization algorithms that can be used to train the disclosed AI system are Momentum, Nesterov accelerated gradient, Adagrad, Adadelta, RMSprop, Adam, AdaMax, Nadam, and AMSGrad.

### Transformer Logic

Machine learning is the use and development of computer systems that can learn and adapt without following explicit instructions, by using algorithms and statistical models to analyze and draw inferences from patterns in data. Some of the state-of-the-art models use Transformers, a more powerful and faster model than neural networks alone. Transformers originate from the field of natural language processing (NLP), but can be used in computer vision and many other fields. Neural networks process input in series and weight relationships by distance in the series. Transformers can process input in parallel and do not necessarily weigh by distance. For example, in natural language processing, neural networks process a sentence from beginning to end with the weights of words close to each other being higher than those further apart. This leaves the end of the sentence very disconnected from the beginning causing an effect called the vanishing gradient problem. Transformers look at each word in parallel and determine weights for the relationships to each of the other words in the sentence. These relationships are called hidden states because they are later condensed for use into one vector called the context vector. Transformers can be used in addition to neural networks. This architecture is described here.

### Encoder-Decoder Architecture

FIG. 10 is a schematic representation of an encoder-decoder architecture. This architecture is often used for NLP and has two main building blocks. The first building block is the encoder that encodes an input into a fixed-size vector. In the system we describe here, the encoder is based on a recurrent neural network (RNN). At each time step, t, a hidden state of time step, t−1, is combined with the input value at time step t to compute the hidden state at timestep t. The hidden state at the last time step, encoded in a context vector, contains relationships encoded at all previous time steps. For NLP, each step corresponds to a word. Then the context vector contains information about the grammar and the sentence structure. The context vector can be considered a low-dimensional representation of the entire input space. For NLP, the input space is a sentence, and a training set consists of many sentences.

The context vector is then passed to the second building block, the decoder. For translation, the decoder has been trained on a second language. Conditioned on the input context vector, the decoder generates an output sequence. At each time step, t, the decoder is fed the hidden state of time step, t−1, and the output generated at time step, t−1. The first hidden state in the decoder is the context vector, generated by the encoder. The context vector is used by the decoder to perform the translation.

The whole model is optimized end-to-end by using backpropagation, a method of training a neural network in which the initial system output is compared to the desired output and the system is adjusted until the difference is minimized. In backpropagation, the encoder is trained to extract the right information from the input sequence, the decoder is trained to capture the grammar and vocabulary of the output language. This results in a fluent model that uses context and generalizes well. When training an encoder-decoder model, the real output sequence is used to train the model to prevent mistakes from stacking. When testing the model, the previously predicted output value is used to predict the next one.

When performing a translation task using the encoder-decoder architecture, all information about the input sequence is forced into one vector, the context vector. Information connecting the beginning of the sentence with the end is lost, the vanishing gradient problem. Also, different parts of the input sequence are important for different parts of the output sequence, information that cannot be learned using only RNNs in an encoder-decoder architecture.

### Attention Mechanism

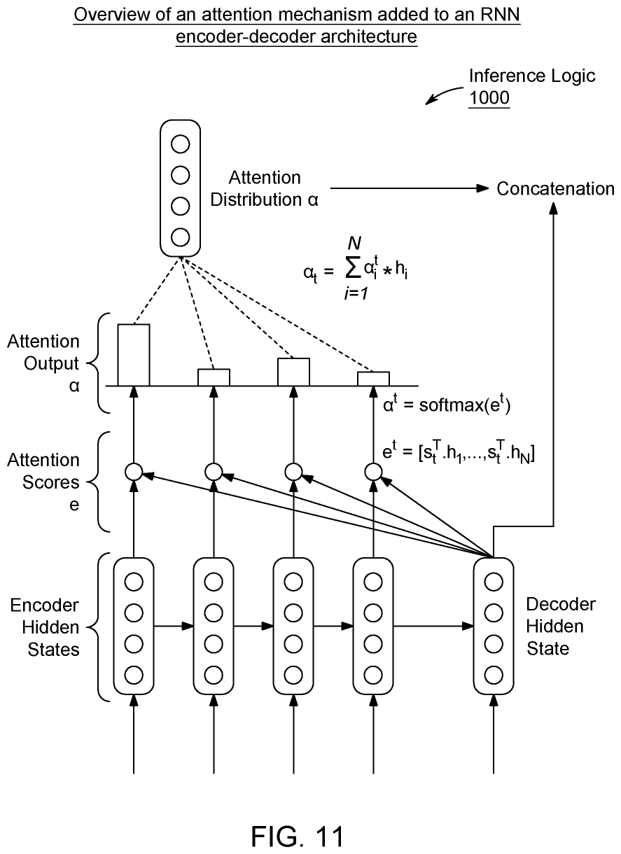

Attention mechanisms distinguish Transformers from other machine learning models. The attention mechanism provides a solution for the vanishing gradient problem. FIG. 11 shows an overview of an attention mechanism added onto an RNN encoder-decoder architecture. At every step, the decoder is given an attention score, e, for each encoder hidden state. In other words, the decoder is given weights for each relationship between words in a sentence. The decoder uses the attention score concatenated with the context vector during decoding. The output of the decoder at time step t is based on all encoder hidden states and the attention outputs. The attention output captures the relevant context for time step t from the original sentence. Thus, words at the end of a sentence may now have a strong relationship with words at the beginning of the sentence. In the sentence “The quick brown fox, upon arriving at the doghouse, jumped over the lazy dog,” fox and dog can be closely related despite being far apart in this complex sentence.

To weight encoder hidden states, a dot product between the decoder hidden state of the current time step, and all encoder hidden states, is calculated. This results in an attention score for every encoder hidden state. The attention scores are higher for those encoder hidden states that are similar to the decoder hidden state of the current time step. Higher values for the dot product indicate the vectors are pointing more closely in the same direction. The attention scores are converted to fractions that sum to one using the SoftMax function.

The SoftMax scores provide an attention distribution. The x-axis of the distribution is position in a sentence. The y-axis is attention weight. The scores show which encoder hidden states are most closely related. The SoftMax scores specify which encoder hidden states are the most relevant for the decoder hidden state of the current time step.

The elements of the attention distribution are used as weights to calculate a weighted sum over the different encoder hidden states. The outcome of the weighted sum is called the attention output. The attention output is used to predict the output, often in combination (concatenation) with the decoder hidden states. Thus, both information about the inputs, as well as the already generated outputs, can be used to predict the next outputs.

By making it possible to focus on specific parts of the input in every decoder step, the attention mechanism solves the vanishing gradient problem. By using attention, information flows more directly to the decoder. It does not pass through many hidden states. Interpreting the attention step can give insights into the data. Attention can be thought of as a soft alignment. The words in the input sequence with a high attention score align with the current target word. Attention describes long-range dependencies better than RNN alone. This enables analysis of longer, more complex sentences.

The attention mechanism can be generalized as: given a set of vector values and a vector query, attention is a technique to compute a weighted sum of the vector values, dependent on the vector query. The vector values are the encoder hidden states, and the vector query is the decoder hidden state at the current time step.

The weighted sum can be considered a selective summary of the information present in the vector values. The vector query determines on which of the vector values to focus. Thus, a fixed-size representation of the vector values can be created, in dependence upon the vector query.

The attention scores can be calculated by the dot product, or by weighing the different values (multiplicative attention).

### Embeddings

For most machine learning models, the input to the model needs to be numerical. The input to a translation model is a sentence, and words are not numerical. multiple methods exist for the conversion of words into numerical vectors. These numerical vectors are called the embeddings of the words. Embeddings can be used to convert any type of symbolic representation into a numerical one.

Embeddings can be created by using one-hot encoding. The one-hot vector representing the symbols has the same length as the total number of possible different symbols. Each position in the one-hot vector corresponds to a specific symbol. For example, when converting colors to a numerical vector, the length of the one-hot vector would be the total number of different colors present in the dataset. For each input, the location corresponding to the color of that value is one, whereas all the other locations are valued at zero. This works well for working with images. For NLP, this becomes problematic, because the number of words in a language is very large. This results in enormous models and the need for a lot of computational power. Furthermore, no specific information is captured with one-hot encoding. From the numerical representation, it is not clear that orange and red are more similar than orange and green. For this reason, other methods exist.

A second way of creating embeddings is by creating feature vectors. Every symbol has its specific vector representation, based on features. With colors, a vector of three elements could be used, where the elements represent the amount of yellow, red, and/or blue needed to create the color. Thus, all colors can be represented by only using a vector of three elements. Also, similar colors have similar representation vectors.

For NLP, embeddings based on context, as opposed to words, are small and can be trained. The reasoning behind this concept is that words with similar meanings occur in similar contexts. Different methods take the context of words into account. Some methods, like GloVe, base their context embedding on co-occurrence statistics from corpora (large texts) such as Wikipedia. Words with similar co-occurrence statistics have similar word embeddings. Other methods use neural networks to train the embeddings. For example, they train their embeddings to predict the word based on the context (Common Bag of Words), and/or to predict the context based on the word (Skip-Gram). Training these contextual embeddings is time intensive. For this reason, pre-trained libraries exist. Other deep learning methods can be used to create embeddings. For example, the latent space of a variational autoencoder (VAE) can be used as the embedding of the input. Another method is to use 1D convolutions to create embeddings. This causes a sparse, high-dimensional input space to be converted to a denser, low-dimensional feature space.

### Self-Attention: Queries (Q), Keys (K), Values (V)

Transformer models are based on the principle of self-attention. Self-attention allows each element of the input sequence to look at all other elements in the input sequence and search for clues that can help it to create a more meaningful encoding. It is a way to look at which other sequence elements are relevant for the current element. The Transformer can grab context from both before and after the currently processed element.

When performing self-attention, three vectors need to be created for each element of the encoder input: the query vector (Q), the key vector (K), and the value vector (V). These vectors are created by performing matrix multiplications between the input embedding vectors using three unique weight matrices.

After this, self-attention scores are calculated. When calculating self-attention scores for a given element, the dot products between the query vector of this element and the key vectors of all other input elements are calculated. To make the model mathematically more stable, these self-attention scores are divided by the root of the size of the vectors. This has the effect of reducing the importance of the scalar thus emphasizing the importance of the direction of the vector. Just as before, these scores are normalized with a SoftMax layer. This attention distribution is then used to calculate a weighted sum of the value vectors, resulting in a vector z for every input element. In the attention principle explained above, the vector to calculate attention scores and to perform the weighted sum was the same, in self-attention two different vectors are created and used. As the self-attention needs to be calculated for all elements (thus a query for every element), one formula can be created to calculate a Z matrix. The rows of this Z matrix are the z vectors for every sequence input element, giving the matrix a size length sequence dimension QKV.

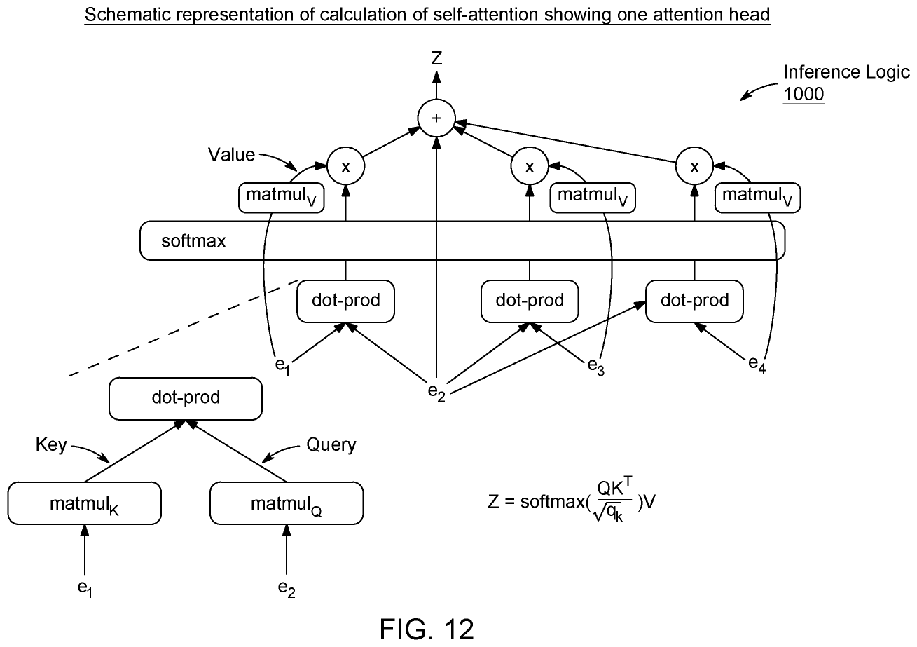

Multi-headed attention is executed in the Transformer. FIG. 12 is a schematic representation of the calculation of self-attention showing one attention head. For every attention head, different weight matrices are trained to calculate Q, K, and V. Every attention head outputs a matrix Z. Different attention heads can capture different types of information. The different Z matrices of the different attention heads are concatenated. This matrix can become large when multiple attention heads are used. To reduce dimensionality, an extra weight matrix W is trained to condense the different attention heads into a matrix with the same size as one Z matrix. This way, the amount of data given to the next step does not enlarge every time self-attention is performed.

When performing self-attention, information about the order of the different elements within the sequence is lost. To address this problem, positional encodings are added to the embedding vectors. Every position has its unique positional encoding vector. These vectors follow a specific pattern, which the Transformer model can learn to recognize. This way, the model can consider distances between the different elements.

As discussed above, in the core of self-attention are three objects: queries (Q), keys (K), and values (V). Each of these objects has an inner semantic meaning of their purpose. One can think of these as analogous to databases. We have a user-defined query of what the user wants to know. Then we have the relations in the database, i.e., the values which are the weights. More advanced database management systems create some apt representation of its relations to retrieve values more efficiently from the relations. This can be achieved by using indexes, which represent information about what is stored in the database. In the context of attention, indexes can be thought of as keys. So instead of running the query against values directly, the query is first executed on the indexes to retrieve where the relevant values or weights are stored. Lastly, these weights are run against the original values to retrieve data that is most relevant to the initial query.

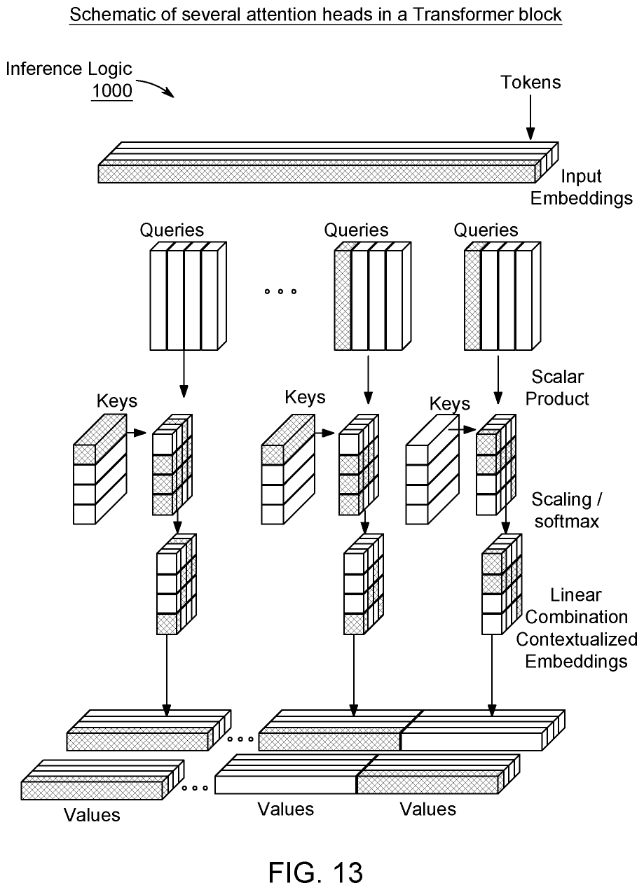

FIG. 13 depicts several attention heads in a Transformer block. We can see that the outputs of queries and keys dot products in different attention heads are differently colored. This depicts the capability of the multi-head attention to focus on different aspects of the input and aggregate the obtained information by multiplying the input with different attention weights.

Examples of attention calculation include scaled dot-product attention and additive attention. There are several reasons why scaled dot-product attention is used in the Transformers. Firstly, the scaled dot-product attention is relatively fast to compute, since its main parts are matrix operations that can be run on modern hardware accelerators. Secondly, it performs similarly well for smaller dimensions of the K matrix, dk, as the additive attention. For larger dk, the scaled dot-product attention performs a bit worse because dot products can cause the vanishing gradient problem. This is compensated via the scaling factor, which is defined as \sqrt{dk}.

As discussed above, the attention function takes as input three objects: key, value, and query. In the context of Transformers, these objects are matrices of shapes (n, d), where n is the number of elements in the input sequence and d is the hidden representation of each element (also called the hidden vector). Attention is then computed as:

$$Attention ( Q , K , V ) = SoftMax ( \frac{Q {K}^{T}}{\sqrt{d k}} ) V$$

where Q, K, V are computed as:

$$X \cdot {W}_{Q} , X \cdot {W}_{K} , X \cdot {W}_{V}$$

X is the input matrix and W_{Q}, W_{K}, W_{V} are learned weights to project the input matrix into the representations. The dot products appearing in the attention function are exploited for their geometrical interpretation where higher values of their results mean that the inputs are more similar, i.e., pointing in the geometrical space in the same direction. Since the attention function now works with matrices, the dot product becomes matrix multiplication. The SoftMax function is used to normalize the attention weights into the value of 1 prior to being multiplied by the values matrix. The resulting matrix is used either as input into another layer of attention or becomes the output of the Transformer.

### Multi-Head Attention

Transformers become even more powerful when multi-head attention is used. Queries, keys, and values are computed the same way as above, though they are now projected into h different representations of smaller dimensions using a set of h learned weights. Each representation is passed into a different scaled dot-product attention block called a head. The head then computes its output using the same procedure as described above.

Formally, the multi-head attention is defined as:

$$MultiHeadAttention ( Q , K , V ) = [ {head}_{1} , … , {head}_{h} ] {W}_{0} where$$

$${head}_{1 i} = Attention ( {QW}_{i}^{Q} , {KW}_{i}^{K} , {VW}_{i}^{V} )$$

The outputs of all heads are concatenated together and projected again using the learned weights matrix W₀ to match the dimensions expected by the next block of heads or the output of the Transformer. Using the multi-head attention instead of the simpler scaled dot-product attention enables Transformers to jointly attend to information from different representation subspaces at different positions.

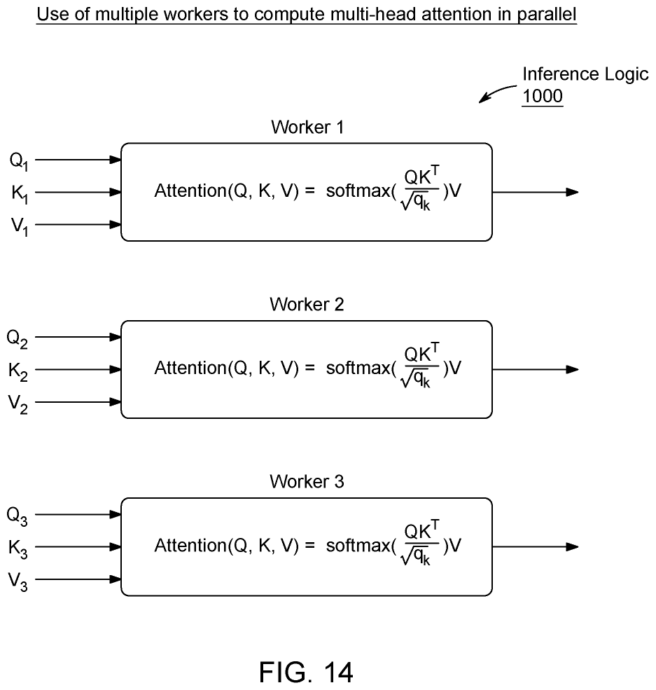

As shown in FIG. 14, one can use multiple workers to compute the multi-head attention in parallel, as the respective heads compute their outputs independently of one another. Parallel processing is one of the advantages of Transformers over RNNs.

Assuming the naive matrix multiplication algorithm which has a complexity of:

$$a \cdot b \cdot c$$

For matrices of shape (a, b) and (c, d), to obtain values Q, K, V, we need to compute the operations:

$$X \cdot {W}_{Q} , X \cdot {W}_{K} , X \cdot WV$$

The matrix X is of shape (n, d) where n is the number of patches and d is the hidden vector dimension. The weights W₀, W_{K}, W_{V} are all of shape (d, d). Omitting the constant factor 3, the resulting complexity is:

$$n \cdot {d}^{2}$$

We can proceed to the estimation of the complexity of the attention function itself, i.e., of

$$SoftMax ( \frac{Q {K}^{T}}{\sqrt{d k}} ) V .$$

The matrices Q and K are both of shape (n, d). The transposition operation does not influence the asymptotic complexity of computing the dot product of matrices of shapes (n, d)·(d, n), therefore its complexity is:

$${n}^{2} \cdot d$$

Scaling by a constant factor of \sqrt{dk}, where dk is the dimension of the keys vector, as well as applying the SoftMax function, both have the complexity of a·b for a matrix of shape (a, b), hence they do not influence the asymptotic complexity. Lastly the dot product

$$SoftMax ( \frac{Q {K}^{T}}{\sqrt{d k}} ) \cdot V$$

is between matrices of shapes (n, n) and (n, d) and so its complexity is:

$${n}^{2} \cdot d$$

The final asymptotic complexity of scaled dot-product attention is obtained by summing the complexities of computing Q, K, V, and of the following attention function:

$$n \cdot {d}^{2} + {n}^{2} \cdot d .$$

The asymptotic complexity of multi-head attention is the same since the original input matrix X is projected into h matrices of shapes

$$( n , \frac{d}{h} ) ,$$

where h is the number of heads. From the point of view of asymptotic complexity, h is constant, therefore we would arrive at the same estimate of asymptotic complexity using a similar approach as for the scaled dot-product attention.

Transformer models often have the encoder-decoder architecture, although this is not necessarily the case. The encoder is built out of different encoder layers which are all constructed in the same way. The positional encodings are added to the embedding vectors. Afterward, self-attention is performed.

### Encoder Block of Transformer

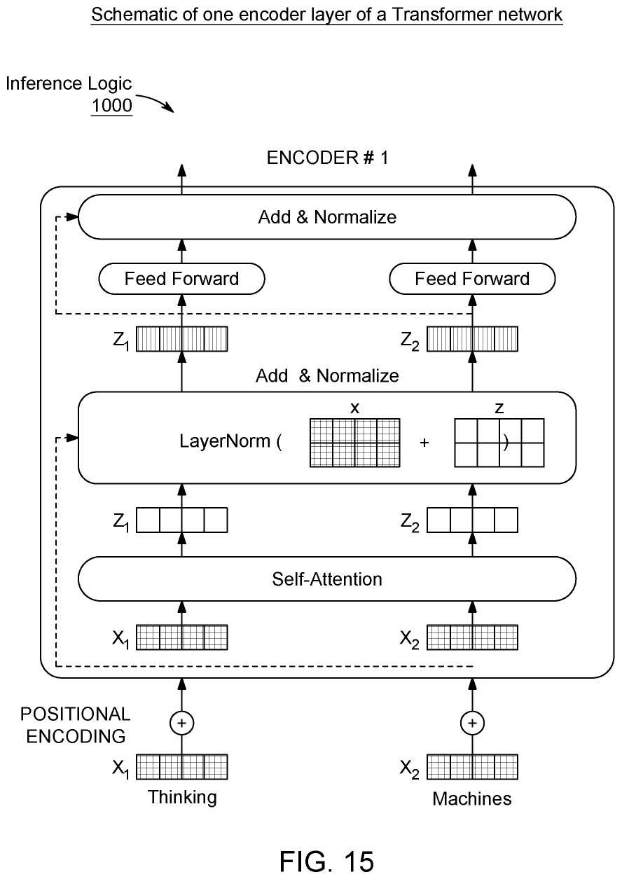

FIG. 15 portrays one encoder layer of a Transformer network. Every self-attention layer is surrounded by a residual connection, summing up the output and input of the self-attention. This sum is normalized, and the normalized vectors are fed to a feed-forward layer. Every z vector is fed separately to this feed-forward layer. The feed-forward layer is wrapped in a residual connection and the outcome is normalized too. Often, numerous encoder layers are piled to form the encoder. The output of the encoder is a fixed-size vector for every element of the input sequence.

Just like the encoder, the decoder is built from different decoder layers. In the decoder, a modified version of self-attention takes place. The query vector is only compared to the keys of previous output sequence elements. The elements further in the sequence are not known yet, as they still must be predicted. No information about these output elements may be used.

### Encoder-Decoder Blocks of Transformer

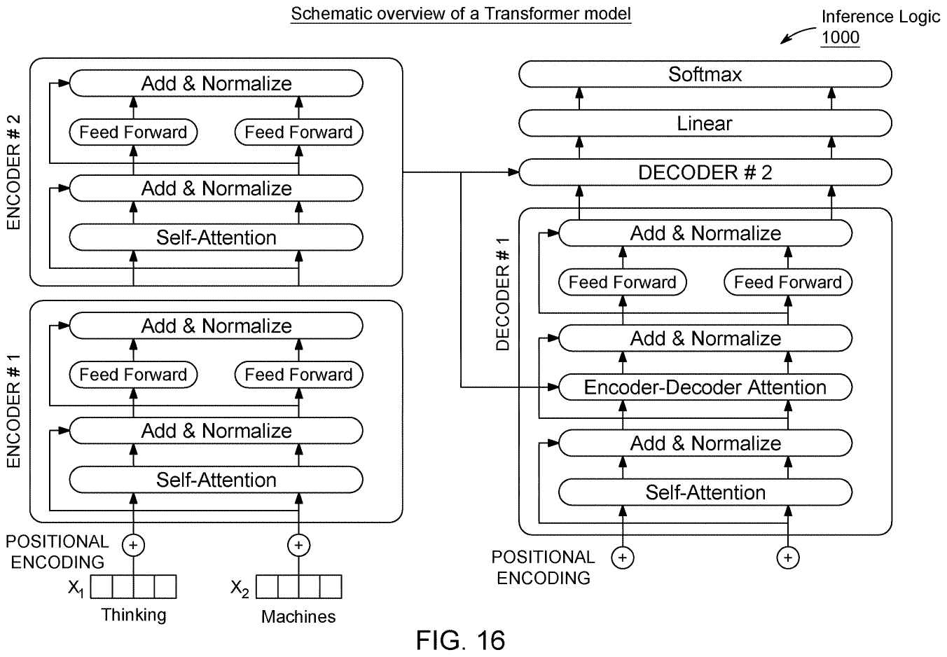

FIG. 16 shows a schematic overview of a Transformer model. Next to a self-attention layer, a layer of encoder-decoder attention is present in the decoder, in which the decoder can examine the last Z vectors of the encoder, providing fluent information transmission. The ultimate decoder layer is a feed-forward layer. All layers are packed in a residual connection. This allows the decoder to examine all previously predicted outputs and all encoded input vectors to predict the next output. Thus, information from the encoder is provided to the decoder, which could improve the predictive capacity. The output vectors of the last decoder layer need to be processed to form the output of the entire system. This is done by a combination of a feed-forward layer and a SoftMax function. The output corresponding to the highest probability is the predicted output value for a subject time step.

For some tasks other than translation, only an encoder is needed. This is true for both document classification and name entity recognition. In these cases, the encoded input vectors are the input of the feed-forward layer and the SoftMax layer. Transformer models have been extensively applied in different NLP fields, such as translation, document summarization, speech recognition, and named entity recognition. These models have applications in the field of biology as well for predicting protein structure and function and labeling DNA sequences.

### Vision Transformer

There are extensive applications of transformers in vision including popular recognition tasks (e.g., image classification, object detection, action recognition, and segmentation), generative modeling, multi-modal tasks (e.g., visual-question answering, visual reasoning, and visual grounding), video processing (e.g., activity recognition, video forecasting), low-level vision (e.g., image super-resolution, image enhancement, and colorization) and 3D analysis (e.g., point cloud classification and segmentation).

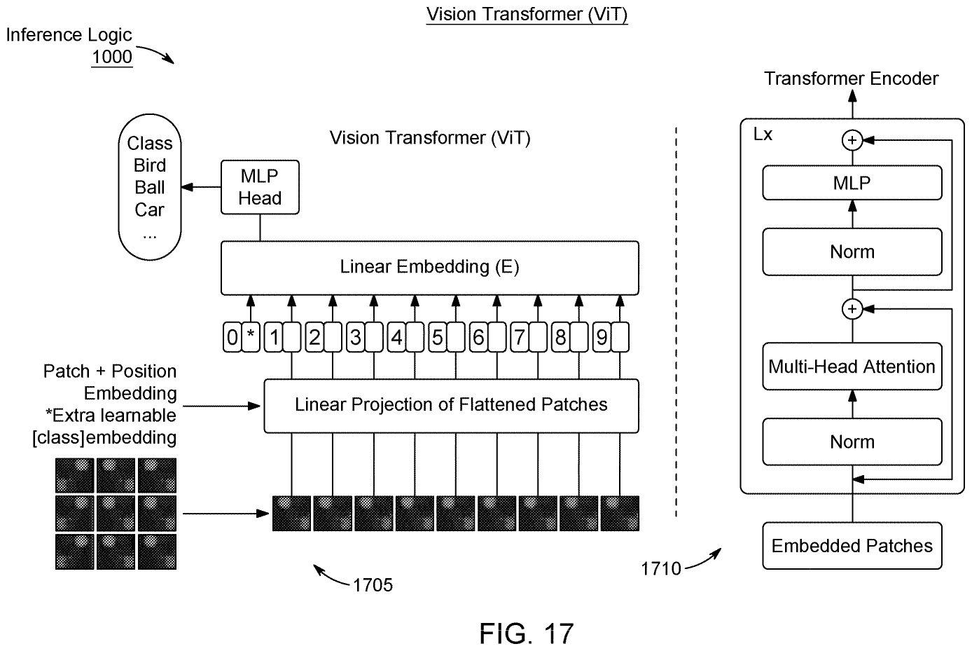

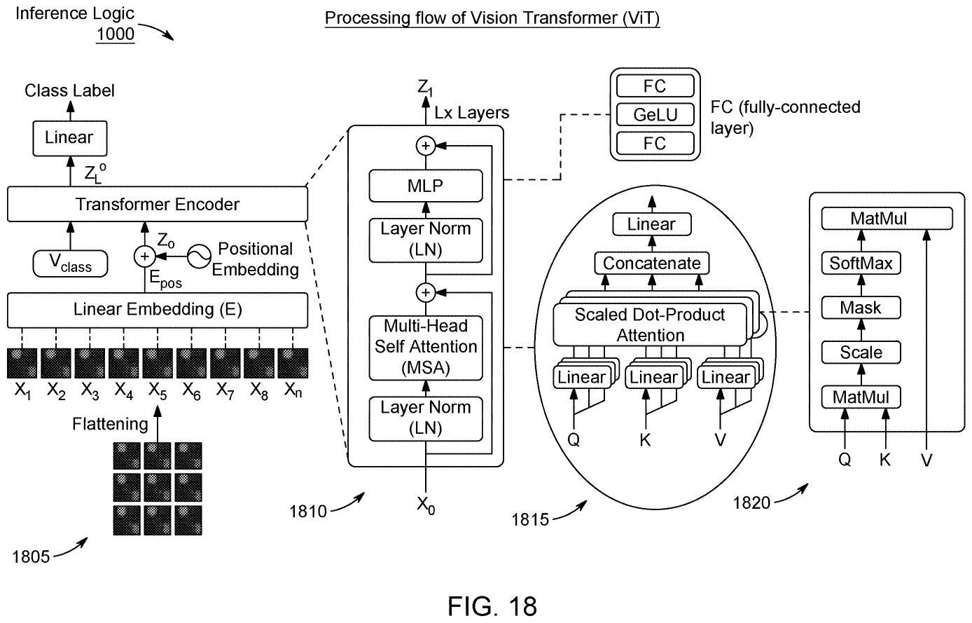

Transformers were originally developed for NLP and worked with sequences of words. In image classification, we often have a single input image in which the pixels are in a sequence. To reduce the computation required, Vision Transformers (ViTs) cut the input image into a set of fixed-sized patches of pixels. The patches are often 16×16 pixels. They are treated much like words in NLP Transformers. ViTs are depicted in FIG. 17 (1705, 1710) and FIG. 18 (1805, 1810, 1815, 1820). Unfortunately, important positional information is lost because image sets are position-invariant. This problem is solved by adding a learned positional encoding into the image patches.

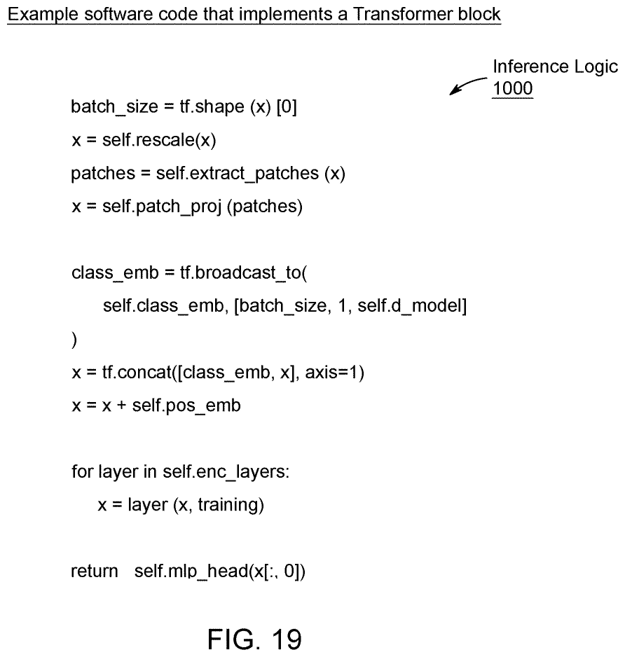

The computations of the ViT architecture can be summarized as follows. The first layer of a ViT extracts a fixed number of patches from an input image. The patches are then projected to linear embeddings. A special class token vector is added to the sequence of embedding vectors to include all representative information of all tokens through the multi-layer encoding procedure. The class vector is unique to each image. Vectors containing positional information are combined with the embeddings and the class token. The sequence of embedding vectors is passed into the Transformer blocks. The class token vector is extracted from the output of the last Transformer block and is passed into a multilayer perceptron (MLP) head whose output is the final classification. The perceptron takes the normalized input and places the output in categories. It classifies the images. This procedure directly translates into the Python Keras code shown in FIG. 19.

When the input image is split into patches, a fixed patch size is specified before instantiating a ViT. Given the quadratic complexity of attention, patch size has a large effect on the length of training and inference time. A single Transformer block comprises several layers. The first layer implements Layer Normalization, followed by the multi-head attention that is responsible for the performance of ViTs. In the depiction of a Transformer block, we can see two arrows. These are residual skip connections. Including skip connection data can simplify the output and improve the results. The output of the multi-head attention is followed again by Layer Normalization. And finally, the output layer is an MLP (Multi-Layer Perceptron) with the GELU (Gaussian Error Linear Unit) activation function.

ViTs can be pretrained and fine-tuned. Pretraining is generally done on a large dataset. Fine-tuning is done on a domain specific dataset.

Domain-specific architectures, like convolutional neural networks (CNNs) or long short-term memory networks (LSTMs), have been derived from the usual architecture of MLPs and suffer from so-called inductive biases that predispose the networks towards a certain output. ViTs stepped in the opposite direction of CNNs and LSTMs and became more general architectures by eliminating inductive biases. A ViT can be seen as a generalization of MLPs because MLPs, after being trained, do not change their weights for different inputs. On the other hand, ViTs compute their attention weights at runtime based on the particular input.

### CLAUSES

The technology disclosed can be practiced as a system, method, or article of manufacture. One or more features of an implementation can be combined with the base implementation. Implementations that are not mutually exclusive are taught to be combinable. One or more features of an implementation can be combined with other implementations. This disclosure periodically reminds the user of these options. Omission from some implementations of recitations that repeat these options should not be taken as limiting the combinations taught in the preceding sections—these recitations are hereby incorporated forward by reference into each of the following implementations.

One or more implementations and clauses of the technology disclosed, or elements thereof can be implemented in the form of a computer product, including a non-transitory computer readable storage medium with computer usable program code for performing the method steps indicated. Furthermore, one or more implementations and clauses of the technology disclosed, or elements thereof can be implemented in the form of an apparatus including a memory and at least one processor that is coupled to the memory and operative to perform exemplary method steps. Yet further, in another aspect, one or more implementations and clauses of the technology disclosed or elements thereof can be implemented in the form of means for carrying out one or more of the method steps described herein; the means can include (i) hardware module(s), (ii) software module(s) executing on one or more hardware processors, or (iii) a combination of hardware and software modules; any of (i)-(iii) implement the specific techniques set forth herein, and the software modules are stored in a computer readable storage medium (or multiple such media).

The clauses described in this section can be combined as features. In the interest of conciseness, the combinations of features are not individually enumerated and are not repeated with each base set of features. The reader will understand how features identified in the clauses described in this section can readily be combined with sets of base features identified as implementations in other sections of this application. These clauses are not meant to be mutually exclusive, exhaustive, or restrictive; and the technology disclosed is not limited to these clauses but rather encompasses all possible combinations, modifications, and variations within the scope of the claimed technology and its equivalents.

Other implementations of the clauses described in this section can include a non-transitory computer readable storage medium storing instructions executable by a processor to perform any of the clauses described in this section. Yet another implementation of the clauses described in this section can include a system including memory and one or more processors operable to execute instructions stored in the memory, to perform any of the clauses described in this section.

### CLAUSES

1. A system comprising one or more processors and memory storing instructions executable by the one or more processors to: receive, in response to a query, from individual generative language models of a plurality of generative language models, a probability distribution over a set of candidate answers; combine the probability distributions into a combined probability distribution by a weighted sum in logarithmic space under expert weights; and emit a final answer based at least in part on the combined probability distribution.
2. A method comprising: obtaining, in response to a query and from individual generative language models of a plurality of generative language models, a probability distribution over a set of candidate answers; combining the probability distributions into a combined probability distribution by a weighted sum of the probability distributions in logarithmic space under expert weights; and emitting a final answer based at least in part on the combined probability distribution.
3. One or more non-transitory computer-readable storage media storing instructions executable by one or more processors to combine a plurality of probability distributions over a set of candidate answers into a combined probability distribution by a weighted sum in logarithmic space under expert weights and to determine a final answer based at least in part on the combined probability distribution.
4. The system of clause 1, wherein the final answer is emitted by a model that is separate from the plurality of generative language models.
5. The system of clause 1, wherein the plurality of generative language models is selected by a triage model based on a task type of the query.
6. The system of clause 1, wherein the expert weights are positive and sum to one.
7. The system of clause 1, wherein the final answer is determined based at least in part on a rationale received from individual generative language models of the plurality of generative language models.
8. The system of clause 1, wherein individual generative language models of the plurality of generative language models are independently substitutable.
9. The system of clause 1, wherein at least one generative language model of the plurality processes the query within an infrastructure that retains the query.
10. The system of clause 1, wherein the combined probability distribution is normalized by a normalization function across the set of candidate answers.
11. The system of clause 1, wherein a final probability distribution determined from the combined probability distribution has a confidence that corresponds to an observed accuracy more closely than a probability distribution of any single generative language model of the plurality.
12. The system of clause 1, wherein the final answer and a representation of a distribution over the set of candidate answers are provided to a computing device.
13. The system of clause 1, wherein the query comprises one of a medical query, a technical query, a financial query, or a classification query.
14. The system of clause 1, wherein a contribution of an individual generative language model to the combining is adjustable.
15. The method of clause 2, further comprising reweighting the combined probability distribution based on a frequency with which individual candidate answers appear at individual ranks across the probability distributions.
16. The method of clause 2, wherein the combining computes a weighted geometric combination of the probability distributions.
17. The method of clause 2, further comprising determining, by a triage model, a task type from the query and selecting the plurality of generative language models based on the task type.
18. The method of clause 2, wherein at least one generative language model of the plurality processes the query within an infrastructure that retains the query.
19. The method of clause 2, further comprising emitting the final answer by a model that is separate from the plurality of generative language models.
20. The method of clause 2, further comprising providing the final answer and a representation of a distribution over the set of candidate answers to a computing device.
21. A system comprising one or more processors and memory storing instructions executable by the one or more processors to: determine, by a triage model and based on a query, a task type and a plurality of specialties, and map individual specialties to corresponding generative language models of a plurality of generative language models; receive, from individual generative language models, a probability distribution over a set of candidate answers; compute a combined probability distribution as a weighted sum, in logarithmic space, of the probability distributions under expert weights that are positive and sum to one; normalize the combined probability distribution to a normalized probability distribution; determine a final probability distribution based at least in part on the normalized probability distribution; and emit, by a consensus model that is separate from the plurality of generative language models, a final answer based at least in part on the final probability distribution and on a rationale received from individual generative language models.
22. A method comprising: determining, by a triage model and based on a query, a task type and a plurality of specialties, and mapping individual specialties to corresponding generative language models of a plurality of generative language models; receiving, from individual generative language models, a probability distribution over a set of candidate answers; computing a combined probability distribution as a weighted sum, in logarithmic space, of the probability distributions under expert weights that are positive and sum to one; normalizing the combined probability distribution to a normalized probability distribution; determining a final probability distribution based at least in part on the normalized probability distribution; and emitting, by a consensus model that is separate from the plurality of generative language models, a final answer based at least in part on the final probability distribution and on a rationale received from individual generative language models.
23. The system of clause 21, wherein the final probability distribution is determined by reweighting the normalized probability distribution based on a rank-frequency table that records a frequency of individual candidate answers at individual ranks across the probability distributions.
24. The system of clause 23, wherein the reweighting applies a boost scaler that scales a rank-weighted frequency contribution added to a normalized probability of a candidate answer.
25. The system of clause 21, wherein, when the probability distributions are mutually divergent, the normalized probability distribution is more uniform than when the probability distributions are mutually consistent.
26. The system of clause 21, wherein the consensus model emits the final answer further based on a specialty associated with individual generative language models.
27. The system of clause 21, wherein the consensus model determines a measure of support that rationales provide for individual candidate answers and emits the final answer based at least in part on the measure of support.
28. The system of clause 21, wherein the consensus model emits the final answer independently of which candidate answer has a highest probability in the final probability distribution.
29. The system of clause 21, wherein the triage model adds a generative language model to, or removes a generative language model from, the plurality based on the task type.
30. The system of clause 21, wherein the plurality of generative language models is selected from a model pool to satisfy a configurable objective comprising at least one of a cost objective, a latency objective, or an accuracy objective.
31. The system of clause 21, wherein a generative language model of the plurality is substituted for a different model without reconfiguration of the consensus model, and the set of candidate answers remains defined across the substitution.
32. The system of clause 21, wherein at least one generative language model is a locally hosted model that processes the query without transmission of the query to a service external to a local infrastructure.
33. The system of clause 21, wherein the set of candidate answers comprises a set of mutually exclusive answer options over which individual probability distributions assign probabilities summing to one.
34. The system of clause 21, wherein the final probability distribution has a calibration, between a confidence indicated by the final probability distribution and an observed accuracy, that is improved relative to a probability distribution of any single generative language model of the plurality.
35. The method of clause 22, further comprising constructing a rank-frequency table that records a frequency of individual candidate answers at individual ranks across the probability distributions, and reweighting the normalized probability distribution based on the rank-frequency table to determine the final probability distribution.
36. The method of clause 35, further comprising applying cascade weights that decrease monotonically with rank to a frequency recorded in the rank-frequency table.
37. The method of clause 22, wherein a rationale received from a generative language model comprises at least one of a clinical reasoning, a scientific reasoning, or an elimination reasoning.
38. The method of clause 22, further comprising adjusting an expert weight to vary a contribution of a corresponding generative language model to the combined probability distribution.
39. The method of clause 22, further comprising substituting a generative language model of the plurality for a different model without reconfiguration of the consensus model, the set of candidate answers remaining defined across the substituting.
40. The method of clause 22, further comprising providing the final answer and a representation of the final probability distribution to a computing device as a decision support output.
41. A system comprising one or more processors and memory storing instructions executable by the one or more processors to: determine, by a triage model and based on a query, a task type and a plurality of specialties, and map individual specialties to corresponding generative language models of a plurality of generative language models, at least one generative language model comprising a locally hosted model that processes the query within an infrastructure that retains the query; receive, from individual generative language models, a probability distribution over a set of candidate answers; compute a combined probability distribution as a weighted sum, in logarithmic space, of the probability distributions under expert weights that are positive and sum to one; normalize the combined probability distribution by applying a softmax function across the set of candidate answers to produce a normalized probability distribution; construct a rank-frequency table that records, for individual candidate answers, a frequency of the candidate answer at individual ranks of a plurality of ranks across the probability distributions; compute, for individual candidate answers, a boosted score comprising a sum of a normalized probability of the candidate answer and a product of a boost scaler and a sum, over the plurality of ranks, of the frequency of the candidate answer at a rank weighted by a respective cascade weight of a plurality of cascade weights, a cascade weight at a first rank being one and a cascade weight at each successive rank being one-half of the cascade weight at the immediately preceding rank; normalize the boosted scores by applying a softmax function across the set of candidate answers to produce a final probability distribution; and emit, by a consensus model that is separate from the plurality of generative language models, a final answer based at least in part on the final probability distribution, on a rationale received from individual generative language models, and on a specialty mapped to individual generative language models.
42. A method comprising: determining, by a triage model and based on a query, a task type and a plurality of specialties, and mapping individual specialties to corresponding generative language models of a plurality of generative language models, at least one generative language model comprising a locally hosted model that processes the query within an infrastructure that retains the query; receiving, from individual generative language models, a probability distribution over a set of candidate answers; computing a combined probability distribution as a weighted sum, in logarithmic space, of the probability distributions under expert weights that are positive and sum to one; normalizing the combined probability distribution by applying a softmax function across the set of candidate answers to produce a normalized probability distribution; constructing a rank-frequency table that records, for individual candidate answers, a frequency of the candidate answer at individual ranks of a plurality of ranks across the probability distributions; computing, for individual candidate answers, a boosted score comprising a sum of a normalized probability of the candidate answer and a product of a boost scaler and a sum, over the plurality of ranks, of the frequency of the candidate answer at a rank weighted by a respective cascade weight of a plurality of cascade weights, a cascade weight at a first rank being one and a cascade weight at each successive rank being one-half of the cascade weight at the immediately preceding rank; normalizing the boosted scores by applying a softmax function across the set of candidate answers to produce a final probability distribution; and emitting, by a consensus model that is separate from the plurality of generative language models, a final answer based at least in part on the final probability distribution, on a rationale received from individual generative language models, and on a specialty mapped to individual generative language models.
43. The system of clause 41, wherein the weighted sum in logarithmic space computes, for individual candidate answers, a sum over the plurality of generative language models of a logarithm of a probability assigned to the candidate answer by a generative language model, weighted by an expert weight of the generative language model.
44. The system of clause 41, wherein the plurality of cascade weights comprises the sequence one, one-half, one-quarter, one-eighth, one-sixteenth, and one-thirty-second.
45. The system of clause 41, wherein the boost scaler comprises a tunable parameter that adjusts a relative influence of the product on the boosted score.
46. The system of clause 41, wherein the boosted score increases a probability of a candidate answer that is ranked within a threshold number of highest ranks in two or more of the probability distributions relative to a probability of the candidate answer in the normalized probability distribution.
47. The system of clause 41, wherein the consensus model determines a measure of support derived from at least one of a consistency or a specificity of rationales that favor a candidate answer, and emits the final answer based at least in part on the measure of support independently of which candidate answer has a highest probability in the final probability distribution.
48. The system of clause 41, wherein the locally hosted model comprises an open-source generative language model deployed on the one or more processors within the infrastructure.
49. The system of clause 41, wherein the plurality of generative language models is selected from a model pool to satisfy a configurable objective, a selection favoring a cost objective selecting generative language models of lower computational cost, a selection favoring a latency objective selecting generative language models that produce a result in less time, and a selection favoring an accuracy objective selecting generative language models associated with higher correctness.
50. The system of clause 41, wherein the rank-frequency table comprises a row for individual candidate answers and a column for individual ranks, a cell of the rank-frequency table recording the frequency of a candidate answer at a rank across the probability distributions.
51. The system of clause 41, wherein the one or more processors comprise a plurality of processors that execute the plurality of generative language models in parallel.
52. The system of clause 41, wherein the query comprises a medical query, individual specialties comprise respective medical specialties, and the set of candidate answers comprises a set of mutually exclusive treatment options or diagnoses.
53. The system of clause 41, wherein the triage model, the plurality of generative language models, and the consensus model are stored as respective instructions in the memory and executed by the one or more processors.
54. The method of clause 42, wherein the weighted sum in logarithmic space computes a weighted geometric mean of the probability distributions under the expert weights.
55. The method of clause 42, further comprising assigning the expert weights based on a measure of reliability or a measure of relevance to the task type of individual generative language models.
56. The method of clause 42, further comprising setting the boost scaler to a first value to apply a first boost and to a second value greater than the first value to apply a second boost greater than the first boost.
57. The method of clause 42, further comprising substituting a generative language model of the plurality for a different model without reconfiguration of the consensus model, the set of candidate answers and the final probability distribution remaining defined across the substituting.
58. The method of clause 42, further comprising emitting the final answer as a candidate answer supported by a strongest rationale independently of which candidate answer has a highest probability in the final probability distribution.
59. The method of clause 42, further comprising providing the final answer and a representation of the final probability distribution to a computing device, the representation comprising two or more of the candidate answers ranked according to the final probability distribution.
60. The method of clause 42, wherein, when the probability distributions are mutually divergent, the normalized probability distribution is more uniform than when the probability distributions are mutually consistent.

The above-described hardware description is a non-limiting example of corresponding structure for performing the functionality described herein.

Numerous modifications and variations of the present disclosure are possible in light of the above teachings. It is therefore to be understood that the technology disclosed may be practiced otherwise than as specifically described herein.

1. A system comprising:
one or more processors; and
one or more non-transitory computer-readable storage media storing instructions executable by the one or more processors to perform operations comprising:
determining, by a triage model and based on a query, a task type and a plurality of specialties, and mapping individual specialties of the plurality of specialties to corresponding expert models of a plurality of expert models, individual expert models of the plurality of expert models comprising respective generative language models, and at least one expert model of the plurality of expert models comprising a locally hosted model that processes the query within an infrastructure that retains the query;
receiving, from individual expert models of the plurality of expert models, a probability distribution over a set of candidate answers, individual expert models of the plurality of expert models being independently configured to process the query according to a mapped specialty;
computing a combined probability distribution over the set of candidate answers as a weighted sum, in logarithmic space, of the probability distributions received from the plurality of expert models, the weighted sum applying a respective expert weight to individual probability distributions, the expert weights being positive and summing to one;
normalizing the combined probability distribution to produce a normalized probability distribution, the normalized probability distribution assigning a normalized probability to individual candidate answers of the set of candidate answers;
determining a final probability distribution over the set of candidate answers based at least in part on the normalized probability distribution;
emitting, by a consensus model that is separate from the plurality of expert models, and based at least in part on the final probability distribution and on a respective rationale received from individual expert models of the plurality of expert models, a final answer to the query; and
providing the final answer and a representation of the final probability distribution to a computing device as a decision support output.

2. The system of claim 1, wherein determining the final probability distribution comprises:
constructing a rank-frequency table that records, for individual candidate answers of the set of candidate answers, a frequency of the candidate answer at individual ranks of a plurality of ranks across the probability distributions received from the plurality of expert models;
computing, for individual candidate answers of the set of candidate answers, a boosted score comprising a sum of the normalized probability of the candidate answer and a rank-frequency contribution, the rank-frequency contribution comprising a product of a boost scaler and a sum, over the plurality of ranks, of the frequency of the candidate answer at the individual rank weighted by a respective cascade weight of a plurality of cascade weights that decrease monotonically with rank; and
normalizing the boosted scores to produce the final probability distribution.

3. The system of claim 2, wherein the normalizing of the combined probability distribution comprises applying a softmax function across the set of candidate answers, a cascade weight applied at a first rank is one, individual cascade weights applied at successive ranks are one-half of the cascade weight applied at the immediately preceding rank, the boost scaler comprises a tunable parameter, and the normalizing of the boosted scores comprises applying a softmax function across the set of candidate answers.

4. The system of claim 2, wherein the normalizing of the boosted scores increases a probability of a candidate answer that is ranked within a threshold number of highest ranks in two or more of the probability distributions received from the plurality of expert models relative to a probability of the candidate answer in the normalized probability distribution.

5. The system of claim 1, wherein the final probability distribution has a calibration, between a confidence indicated by the final probability distribution and an observed accuracy, that is improved relative to the probability distribution received from any single expert model of the plurality of expert models.

6. The system of claim 1, wherein the respective generative language models are independently substitutable, and the operations further comprise substituting one expert model of the plurality of expert models for a different model without reconfiguration of the consensus model, the final probability distribution remaining defined over the set of candidate answers after the substituting.

7. The system of claim 1, wherein the locally hosted model processes the query without transmission of the query to a service external to the infrastructure, and the plurality of expert models is selected from a model pool to satisfy a configurable objective comprising at least one of a cost objective, a latency objective, or an accuracy objective.

8. The system of claim 1, wherein mapping individual specialties to corresponding expert models comprises adding an expert model to, or removing an expert model from, the plurality of expert models based on the task type determined by the triage model.

9. The system of claim 1, wherein the respective expert weight applied to individual probability distributions is adjustable to vary a contribution of the corresponding expert model to the combined probability distribution, and the set of candidate answers comprises a set of mutually exclusive answer options over which individual probability distributions assign probabilities summing to one.

10. The system of claim 1, wherein the consensus model emits the final answer further based on a respective specialty mapped to individual expert models of the plurality of expert models and on a strength of the respective rationale received from individual expert models, independently of which candidate answer has a highest probability in the final probability distribution.

11. The system of claim 1, wherein the respective rationale received from individual expert models comprises at least one of a clinical reasoning, a scientific reasoning, or an elimination reasoning, and the query comprises a medical query processed within a clinical decision support workflow.

12. A method comprising:
determining, by one or more processors executing a triage model and based on a query, a task type and a plurality of specialties, and mapping individual specialties of the plurality of specialties to corresponding expert models of a plurality of expert models, individual expert models of the plurality of expert models comprising respective generative language models, and at least one expert model of the plurality of expert models comprising a locally hosted model that processes the query within an infrastructure that retains the query;
receiving, by the one or more processors and from individual expert models of the plurality of expert models, a probability distribution over a set of candidate answers, individual expert models of the plurality of expert models being independently configured to process the query according to a mapped specialty;
computing, by the one or more processors, a combined probability distribution over the set of candidate answers as a weighted sum, in logarithmic space, of the probability distributions received from the plurality of expert models, the weighted sum applying a respective expert weight to individual probability distributions, the expert weights being positive and summing to one;
normalizing, by the one or more processors, the combined probability distribution to produce a normalized probability distribution, the normalized probability distribution assigning a normalized probability to individual candidate answers of the set of candidate answers;
determining, by the one or more processors, a final probability distribution over the set of candidate answers based at least in part on the normalized probability distribution;
emitting, by the one or more processors executing a consensus model that is separate from the plurality of expert models, and based at least in part on the final probability distribution and on a respective rationale received from individual expert models of the plurality of expert models, a final answer to the query; and
providing, by the one or more processors, the final answer and a representation of the final probability distribution to a computing device as a decision support output.

13. The method of claim 12, wherein determining the final probability distribution comprises:
constructing a rank-frequency table that records, for individual candidate answers of the set of candidate answers, a frequency of the candidate answer at individual ranks of a plurality of ranks across the probability distributions received from the plurality of expert models;
computing, for individual candidate answers of the set of candidate answers, a boosted score comprising a sum of the normalized probability of the candidate answer and a product of a boost scaler and a sum, over the plurality of ranks, of the frequency of the candidate answer at the individual rank weighted by a respective cascade weight of a plurality of cascade weights that decrease monotonically with rank; and
normalizing the boosted scores to produce the final probability distribution.

14. The method of claim 13, wherein a cascade weight at a first rank is one, individual cascade weights at successive ranks are one-half of the cascade weight at the immediately preceding rank, the boost scaler comprises a tunable parameter, and the normalizing of the combined probability distribution and the normalizing of the boosted scores each comprise applying a softmax function across the set of candidate answers.

15. The method of claim 12, wherein the locally hosted model processes the query without transmission of the query to a service external to the infrastructure, and the method further comprises substituting one expert model of the plurality of expert models for a different model without reconfiguration of the consensus model.

16. The method of claim 12, wherein emitting the final answer comprises emitting the final answer further based on a respective specialty mapped to individual expert models and on a strength of the respective rationale, independently of which candidate answer has a highest probability in the final probability distribution, and the final probability distribution has a calibration, between a confidence indicated by the final probability distribution and an observed accuracy, that is improved relative to the probability distribution received from any single expert model of the plurality of expert models.

17. The method of claim 12, wherein mapping individual specialties to corresponding expert models comprises adding an expert model to, or removing an expert model from, the plurality of expert models based on the task type, and the plurality of expert models is selected to satisfy a configurable objective comprising at least one of a cost objective, a latency objective, or an accuracy objective.

18. One or more non-transitory computer-readable storage media storing instructions executable by one or more processors to perform operations comprising:
receiving, from individual expert models of a plurality of expert models that are independently configured to process a query according to respective specialties, a probability distribution over a set of candidate answers, individual expert models of the plurality of expert models comprising respective generative language models, and at least one expert model of the plurality of expert models comprising a locally hosted model that processes the query within an infrastructure that retains the query;
combining the probability distributions received from the plurality of expert models into a combined probability distribution over the set of candidate answers as a weighted sum, in logarithmic space, of the probability distributions under respective expert weights that are positive and sum to one, followed by a normalization that assigns a normalized probability to individual candidate answers of the set of candidate answers;
constructing a rank-frequency table that records, for individual candidate answers of the set of candidate answers, a frequency of the candidate answer at individual ranks of a plurality of ranks across the probability distributions received from the plurality of expert models;
computing, for individual candidate answers of the set of candidate answers, a boosted score based on the normalized probability of the candidate answer and on a product of a boost scaler and a sum, over the plurality of ranks, of the frequency of the candidate answer at the individual rank weighted by a respective cascade weight of a plurality of cascade weights that decrease monotonically with rank;
normalizing the boosted scores to produce a final probability distribution over the set of candidate answers;
emitting, by a consensus model that is separate from the plurality of expert models, and based at least in part on the final probability distribution and on a respective rationale received from individual expert models of the plurality of expert models, a final answer to the query; and
providing the final answer and a representation of the final probability distribution to a computing device as a decision support output.

19. The one or more non-transitory computer-readable storage media of claim 18, wherein a cascade weight at a first rank is one, individual cascade weights at successive ranks are one-half of the cascade weight at the immediately preceding rank, the boost scaler comprises a tunable parameter, and the normalizing of the boosted scores comprises applying a softmax function across the set of candidate answers.

20. The one or more non-transitory computer-readable storage media of claim 18, wherein the consensus model emits the final answer further based on a respective specialty mapped to individual expert models of the plurality of expert models and on a strength of the respective rationale, independently of which candidate answer has a highest probability in the final probability distribution, and the operations further comprise substituting one expert model of the plurality of expert models for a different model without reconfiguration of the consensus model.
